(19) United States
(12) Patent Application Publication
(10) Pub. No.: US 2010/0035918 A1
(43) Pub. Date: Feb. 11, 2010

(54) Imidazolone Compounds and Methods of Making and Using the Same

(75) Inventors: Kevin Guckian, Marlborough, MA (US); Wen-Cherng Lee, Lexington, MA (US); Edward Lin, Somerville, MA (US)

Correspondence Address: HONIGMAN MILLER SCHWARTZ & COHN LLP, 444 WEST MICHIGAN AVENUE, KALAMAZOO, MI, 49007-3714, US

(73) Assignee: Biogen Idec MA Inc, Cambridge, MA (US)

(21) Appl. No.: 12/525,106

(22) PCT Filed: Jan. 30, 2008
(86) PCT No.: PCT/US08/01172

§ 371 Date: Oct. 7, 2009

Related U.S. Application Data
(60) Provisional application No. 60/898,441, filed on Jan. 30, 2007.

(51) Int. Cl. A61K 31/437; C07D 471/02; A61P 35/00; A61P 19/04; A61P 1/00; A61P 25/00

(52) U.S. Cl. 514/300; 546/117

(57) ABSTRACT

In one aspect, the invention features a compound of the general Formula (I). Compounds of Formula (I) possess high affinity for Alk 5 and/or AIk 4, and can be useful as antagonists thereof for preventing and/or treating numerous diseases, including fibrotic disorders.

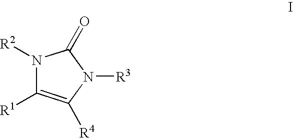

This application claims priority to U.S. Ser. No. 60/898,441, filed on Jan. 30, 2007. The entire contents of the aforementioned application are incorporated herein.

### BACKGROUND OF THE INVENTION

TGFβ (Transforming Growth Factor β) is a member of a large family of dimeric polypeptide growth factors that includes, for example, activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including, for example, interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk 5) in a glycine/serine rich domain, which allows the type I receptor to recruit and phosphorylate downstream signaling molecules, Smad2 or Smad3. See, e.g., Huse, M. et al., Mol. Cell. 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med. 67: 773 (1998).

Activins are also members of the TGFβ superfamily, which are distinct from TGFβ in that they are homo- or heterodimers of activin βa or βb. Activins signal in a manner similar to TGFβ, that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev. Cell. Biol. 6: 594-641 (1990); Roberts, A. B. and Sporn M. B. Peptide Growth Factors and Their Receptors, 95: 419-472 Berlin: Springer-Verlag (1990); Roberts, A. B. and Sporn M. B. Growth Factors 8:1-9 (1993); and Alexandrow, M. G., Moses, H. L. Cancer Res. 55: 1452-1457 (1995).

Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); Sugiyama, M. et al., Gastroenterology 114: 550-558 (1998); Munz, B. et al., EMBO J. 18: 5205-5215 (1999)), inflammatory responses (see, e.g., Rosendahl, A. et al., Am. J. Repir. Cell Mol. Biol. 25: 60-68 (2001)), cachexia or wasting (see Matzuk, M. M. et al., Proc. Nat. Acad. Sci. USA 91: 8817-8821 (1994); Coerver, K. A. et al, Mol. Endocrinol. 10: 534-543 (1996); Cipriano, S. C. et al. Endocrinology 141: 2319-27 (2000)), diseases of or pathological responses in the central nervous system (see Logan, A. et al. Eur. J. Neurosci. 11: 2367-2374 (1999); Logan, A. et al. Exp. Neurol. 159: 504-510 (1999); Masliah, E. et al., Neurochem. Int. 39: 393-400 (2001); De Groot, C. J. A. et al., J. Neuropathol. Exp. Neurol. 58: 174-187 (1999), John, G. R. et al, Nat. Med. 8: 1115-21 (2002)) and hypertension (see Dahly, A. J. et al., Am. J. Physiol. Regul. Integr. Comp. Physiol. 283: R757-67 (2002)).

Studies have shown that TGFβ and activin can act synergistically to induce extracellular matrix production (see, e.g., Sugiyama, M. et al., Gastroenterology 114: 550-558, (1998)). It is therefore desirable to develop modulators (e.g., antagonists) to members of the TGFβ family to prevent and/or treat disorders involving this signaling pathway.

### SUMMARY OF THE INVENTION

The invention is based on the discovery that compounds of Formula (I) are potent antagonists of the TGFβ family type I receptors, Alk5 and/or Alk4. Thus, compounds of Formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

In one aspect, the present invention provides compounds of Formula (I),

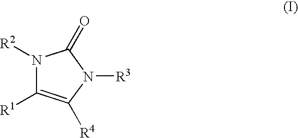

N-oxide derivatives thereof, or pharmaceutically acceptable salts thereof. In Formula (I):

R¹ is an optionally substituted monocyclic heteroaryl containing at least one hetero ring atom selected from the group consisting of O and S, and optionally further containing 1 or 2 N atoms as hetero ring atoms; or R¹ is an optionally substituted monocyclic heteroaryl containing at least 3 N atoms as hetero ring atoms; or R¹ is an optionally substituted 9- to 12-membered bicyclic heteroaryl containing at least 1 ring atom selected from the group consisting of O and S, and optionally also containing 1 to 3 N atoms as hetero ring atoms; or R¹ is an optionally substituted 9- to 12-membered bicyclic heteroaryl containing at least 2 ring atoms each independently selected from the group consisting of O, S, and N; or R¹ is an optionally substituted 10- to 12-membered bicyclic heteroaryl containing at least 1 ring atom each independently selected from the group consisting of O, S, and N;

R² is an optionally substituted aryl or an optionally substituted heteroaryl;

R³ is selected from the group consisting of H, optionally substituted aliphatic, optionally substituted cycloaliphatic, optionally substituted heterocycloaliphatic, optionally substituted araliphatic, optionally substituted heteroaraliphatic, optionally substituted aryl, and optionally substituted heteroaryl; and

R⁴ is selected from the group consisting of H, optionally substituted aliphatic, optionally substituted cycloaliphatic, optionally substituted heterocycloaliphatic, optionally substituted araliphatic, optionally substituted heteroaraliphatic, optionally substituted aryl, and optionally substituted heteroaryl.

In some embodiments, R¹ is an optionally substituted 9- to 12-membered bicyclic heteroaryl containing 2 to 4 ring atoms each independently selected from the group consisting of O, S, and N; or R¹ is an optionally substituted 10- to 12-membered bicyclic heteroaryl containing at least 1 ring atom each independently selected from the group consisting of O, S, and N.

In some embodiments, R¹ is benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, or cinnolinyl; and R¹ is optionally substituted.

In some embodiments, R¹ is optionally substituted [1,2,4]triazolo[1,5-a]pyridin-6-yl.

In some embodiments, R¹ is substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R² is an optionally substituted aryl (e.g., optionally substituted phenyl).

In some embodiments, R² is phenyl and is substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R² is o-, m-, or p-methylphenyl.

In some embodiments, R² is 3-chloro-4-fluorophenyl.

In some embodiments, R² is an optionally substituted heteroaryl.

In some embodiments, R² is an optionally substituted monocyclic heteroaryl.

In some embodiments, R² is optionally substituted pyridinyl or optionally substituted pyrimidinyl.

In some embodiments, R² is an optionally substituted bicyclic heteroaryl.

In some embodiments, R² is selected from the group consisting of benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, and cinnolinyl; and R² is optionally substituted.

In some embodiments, R² is optionally substituted benzo[1,3]dioxolyl.

In some embodiments, R² is substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R³ is selected from the group consisting of H, optionally substituted C₁₋₆ aliphatic, optionally substituted C₃₋₁₀ cycloaliphatic, optionally substituted C₃₋₁₀ heterocycloaliphatic, optionally substituted C₄₋₁₀ araliphatic, optionally substituted C₃₋₁₀ heteroaraliphatic, optionally substituted C₄₋₁₀ aryl and optionally substituted C₃₋₁₀ heteroaryl.

In some embodiments, R³ is methyl substituted with an optionally substituted aryl or an optionally substituted heteroaryl.

In some embodiments, R³ is benzyl and the phenyl moiety in the benzyl group is optionally substituted with 1 to 3 substituents each independently selected from the group consisting of alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R³ is methyl substituted with a heteroaryl selected from the group consisting of benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, and cinnolinyl; and the heteroaryl is optionally substituted.

In some embodiments, the heteroaryl substituent in R³ is optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R³ is an optionally substituted cycloaliphatic of Formula (Ia).

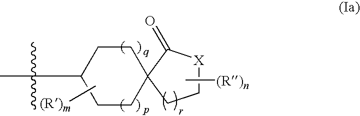

### In Formula (Ia):

X is O or NR^{Q};

R^{Q} is H, C₁₋₄ aliphatic, C₃₋₇ cycloalkyl, C₆₋₁₂ aryl, or C₅₋₁₂ heteroaryl;

each R′ is independently C₁₋₄ aliphatic, halo, cyano, hydroxy, carboxy, amido, amino, or alkoxy;

each R″ is independently C₁₋₄ aliphatic, halo, cyano, hydroxy, carboxy, amido, amino, or alkoxy;

each of p and q is independently 0, 1, or 2, provided the sum of p and q is 2, 3, or 4;

r is 1, 2 or 3; and

each of m and n is independently 0, 1, or 2.

Another aspect of this invention provides compounds of Formula (I),

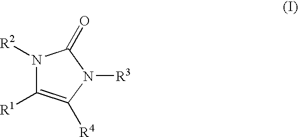

N-oxide derivatives thereof, or pharmaceutically acceptable salts thereof. In Formula (I):

R¹ is an optionally substituted heteroaryl, provided that R¹ is not a monocyclic heteroaryl containing only 1 or 2 N atoms as hetero ring atoms and also that R¹ is not a 9-membered bicyclic heteroaryl containing only 1 N atom as hetero ring atom;

R² is an optionally substituted aryl or an optionally substituted heteroaryl;

R³ is selected from the group consisting of H, optionally substituted aliphatic, optionally substituted cycloaliphatic, optionally substituted heterocycloaliphatic, optionally substituted araliphatic and optionally substituted heteroaraliphatic; and

R⁴ is selected from the group consisting of H, optionally substituted aliphatic, optionally substituted cycloaliphatic, optionally substituted heterocycloaliphatic, optionally substituted araliphatic and optionally substituted heteroaraliphatic.

In some embodiments, R¹ is an optionally substituted 9-membered bicyclic heteroaryl containing 1 ring atom selected from the group consisting of O and S.

In some embodiments, R¹ is an optionally substituted 9- to 12-membered bicyclic heteroaryl containing 2 to 4 ring atoms each independently selected from the group consisting of O, S, and N.

In some embodiments, R² is an optionally substituted aryl.

In some embodiments, R² is an optionally substituted heteroaryl.

In some embodiments, R³ is selected from the group consisting of H, optionally substituted C₁₋₆ aliphatic, optionally substituted C₃₋₁₀ cycloaliphatic, optionally substituted C₃₋₁₀ heterocycloaliphatic, optionally substituted C₄₋₁₀ araliphatic, and optionally substituted C₃₋₁₀ heteroaraliphatic.

In some embodiments, R³ is an optionally substituted cycloaliphatic of Formula (Ia)

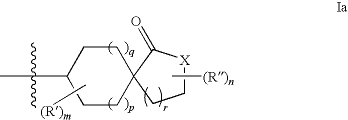

in which:

X is O or NR^{Q};

R^{Q} is H, C₁₋₄ aliphatic, C₃₋₇ cycloalkyl, C₆₋₁₂ aryl, or C₅₋₁₂ heteroaryl;

each R′ is independently C₁₋₄ aliphatic, halo, cyano, hydroxy, carboxy, amido, amino, or alkoxy;

each R″ is independently C₁₋₄ aliphatic, halo, cyano, hydroxy, carboxy, amido, amino, or alkoxy;

each of p and q is independently 0, 1, or 2, provided the sum of p and q is 2, 3, or 4;

r is 1, 2 or 3; and

each of m and n is independently 0, 1, or 2.

In some embodiments, the compound is
3-((4-([1,2,4]triazolo[1,5-a]pyridin-6-yl)-5-methyl-2-oxo-3-m-tolyl-2,3-dihydro-1H-imidazol-1-yl)methyl)benzoic acid;
4-((4-([1,2,4]triazolo[1,5-a]pyridin-6-yl)-5-methyl-2-oxo-3-m-tolyl-2,3-dihydro-1H-imidazol-1-yl)methyl)benzoic acid;
5-([1,2,4]triazolo[1,5-a]pyridin-6-yl)-1-(3-chloro-4-fluorophenyl)-4-methyl-1H-imidazol-2(3H)-one;
4-([1,2,4]triazolo[1,5-a]pyridin-6-yl)-5-methyl-1-(1-oxo-2-oxaspiro[4.5]decan-8-yl)-3-m-tolyl-1H-imidazol-2(3H)-one;
4-([1,2,4]triazolo[1,5-a]pyridin-6-yl)-1-(cyclobutylmethyl)-5-methyl-3-phenyl-1H-imidazol-2(3H)-one;
4-([1,2,4]triazolo[1,5-a]pyridin-6-yl)-1-(2-aminobenzyl)-5-methyl-3-m-tolyl-1H-imidazol-2(3H)-one;
4-([1,2,4]triazolo[1,5-a]pyridin-6-yl)-1-(benzo[c][1,2,5]thiadiazol-5-ylmethyl)-5-methyl-3-m-tolyl-1H-imidazol-2(3H)-one;
4-([1,2,4]triazolo[1,5-a]pyridin-6-yl)-5-methyl-1-(2-methylbenzyl)-3-m-tolyl-1H-imidazol-2(3H)-one;
4-([1,2,4]triazolo[1,5-a]pyridin-6-yl)-5-methyl-1-(2-nitrobenzyl)-3-m-tolyl-1H-imidazol-2(3H)-one;
2-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]triazolo[1,5-a]pyridin-6-yl)-1-(3-methoxybenzyl)-5-methyl-3-m-tolyl-1H-imidazol-2(3H)-one;
4-([1,2,4]triazolo[1,5-a]pyridin-6-yl)-5-methyl-1-(3-methylbenzyl)-3-m-tolyl-1H-imidazol-2(3H)-one;
4-([1,2,4]triazolo[1,5-a]pyridin-6-yl)-1-(3-fluorobenzyl)-5-methyl-3-m-tolyl-1H-imidazol-2(3H)-one;
3-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]triazolo[1,5-a]pyridin-6-yl)-5-methyl-1-((tetrahydro-2H-pyran-4-yl)methyl)-3-m-tolyl-1H-imidazol-2(3H)-one;
4-([1,2,4]triazolo[1,5-a]pyridin-6-yl)-5-methyl-1-((5-methylisoxazol-3-yl)methyl)-3-m-tolyl-1H-imidazol-2(3H)-one;
4-([1,2,4]triazolo[1,5-a]pyridin-6-yl)-1-(benzo[c][1,2,5]oxadiazol-5-ylmethyl)-5-methyl-3-m-tolyl-1H-imidazol-2(3H)-one;
ethyl 2-(4-([1,2,4]triazolo[1,5-a]pyridin-6-yl)-5-methyl-2-oxo-3-m-tolyl-2,3-dihydro-1H-imidazol-1-yl)acetate;
4-([1,2,4]triazolo[1,5-a]pyridin-6-yl)-1-(2-methoxyethyl)-5-methyl-3-m-tolyl-1H-imidazol-2(3H)-one;
4-([1,2,4]triazolo[1,5-a]pyridin-6-yl)-5-methyl-1-(pyridin-3-ylmethyl)-3-m-tolyl-1H-imidazol-2(3H)-one;
4-([1,2,4]triazolo[1,5-a]pyridin-6-yl)-5-methyl-1-(pyridin-2-ylmethyl)-3-m-tolyl-1H-imidazol-2(3H)-one;
4-([1,2,4]triazolo[1,5-a]pyridin-6-yl)-1-(3-aminobenzyl)-5-methyl-3-m-tolyl-1H-imidazol-2(3H)-one;
4-([1,2,4]triazolo[1,5-a]pyridin-6-yl)-1-(cyclohexylmethyl)-5-methyl-3-m-tolyl-1H-imidazol-2(3H)-one;
4-([1,2,4]triazolo[1,5-a]pyridin-6-yl)-1,5-dimethyl-3-m-tolyl-1H-imidazol-2(3H)-one;
3-((4-([1,2,4]triazolo[1,5-a]pyridin-6-yl)-5-methyl-2-oxo-3-m-tolyl-2,3-dihydro-1H-imidazol-1-yl)methyl)benzamide;
methyl 3-((4-([1,2,4]triazolo[1,5-a]pyridin-6-yl)-5-methyl-2-oxo-3-m-tolyl-2,3-dihydro-1H-imidazol-1-yl)methyl)benzoate;
4-([1,2,4]triazolo[1,5-a]pyridin-6-yl)-5-methyl-1-(3-nitrobenzyl)-3-m-tolyl-1H-imidazol-2(3H)-one;
methyl 4-((4-([1,2,4]triazolo[1,5-a]pyridin-6-yl)-5-methyl-2-oxo-3-m-tolyl-2,3-dihydro-1H-imidazol-1-yl)methyl)benzoate;
4-([1,2,4]triazolo[1,5-a]pyridin-6-yl)-1-benzyl-5-methyl-3-m-tolyl-1H-imidazol-2(3H)-one;
4-([1,2,4]triazolo[1,5-a]pyridin-6-yl)-1-(4-methoxybenzyl)-5-methyl-3-m-tolyl-1H-imidazol-2(3H)-one;
5-([1,2,4]triazolo[1,5-a]pyridin-6-yl)-4-methyl-1-m-tolyl-1H-imidazol-2(3H)-one;
5-([1,2,4]triazolo[1,5-a]pyridin-6-yl)-1-m-tolyl-1H-imidazol-2(3H)-one;
4-((4-([1,2,4]triazolo[1,5-a]pyridin-6-yl)-5-methyl-2-oxo-3-m-tolyl-2,3-dihydro-1H-imidazol-1-yl)methyl)benzoic acid;
4-([1,2,4]triazolo[1,5-a]pyridin-6-yl)-3-(3-chloro-4-fluorophenyl)-5-methyl-1-(3-methylbenzyl)-1H-imidazol-2(3H)-one;
4-([1,2,4]triazolo[1,5-a]pyridin-6-yl)-1-(benzo[c][1,2,5]oxadiazol-5-ylmethyl)-3-(3-chloro-4-fluorophenyl)-5-methyl-1H-imidazol-2(3H)-one;
4-([1,2,4]triazolo[1,5-a]pyridin-6-yl)-1-(benzo[c][1,2,5]thiadiazol-5-ylmethyl)-3-(3-chloro-4-fluorophenyl)-5-methyl-1H-imidazol-2(3H)-one;
4-([1,2,4]triazolo[1,5-a]pyridin-6-yl)-3-(3-chloro-4-fluorophenyl)-1-(3-fluorobenzyl)-5-methyl-1H-imidazol-2(3H)-one;
4-([1,2,4]triazolo[1,5-a]pyridin-6-yl)-5-methyl-1-(4-methylbenzyl)-3-m-tolyl-1H-imidazol-2(3H)-one;
4-([1,2,4]triazolo[1,5-a]pyridin-6-yl)-1-(4-fluorobenzyl)-5-methyl-3-m-tolyl-1H-imidazol-2(3H)-one;
4-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]triazolo[1,5-a]pyridin-6-yl)-1-(2-fluorobenzyl)-5-methyl-3-m-tolyl-1H-imidazol-2(3H)-one;
4-([1,2,4]triazolo[1,5-a]pyridin-6-yl)-1-(cyclopentylmethyl)-5-methyl-3-m-tolyl-1H-imidazol-2(3H)-one;
4-([1,2,4]triazolo[1,5-a]pyridin-6-yl)-1-(cyclopropylmethyl)-5-methyl-3-m-tolyl-1H-imidazol-2(3H)-one;
4-([1,2,4]triazolo[1,5-a]pyridin-6-yl)-1-benzoyl-3-(3-chloro-4-fluorophenyl)-5-methyl-1H-imidazol-2(3H)-one;
4-([1,2,4]triazolo[1,5-a]pyridin-6-yl)-3-(3-chloro-4-fluorophenyl)-5-methyl-1-(methylsulfonyl)-1H-imidazol-2(3H)-one;
4-([1,2,4]triazolo[1,5-a]pyridin-6-yl)-3-(3-chloro-4-fluorophenyl)-5-methyl-1-(phenylsulfonyl)-1H-imidazol-2(3H)-one; or
4-([1,2,4]triazolo[1,5-a]pyridin-6-yl)-1-acetyl-3-(3-chloro-4-fluorophenyl)-5-methyl-1H-imidazol-2(3H)-one.

An N-oxide derivative or a pharmaceutically acceptable salt of each of the compounds of Formula (I) is also within the scope of this invention. For example, a nitrogen ring atom of the imidazolone core ring or a nitrogen-containing heterocyclyl substituent can form an oxide in the presence of a suitable oxidizing agent such as m-chloroperbenzoic acid or H₂O₂.

A compound of Formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of Formula (I) can be treated with an acid to form acid addition salts. Examples of such acids include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to those skilled in the art. The acid addition salts can be prepared by treating a compound of Formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of Formula (I) can also be, e.g., in a form of achiral compounds, racemic mixtures, optically active compounds, pure diastereomers, or a mixture of diastereomers.

Compounds of Formula (I) exhibit surprisingly high affinity to the TGFβ family type I receptors, Alk 5 and/or Alk 4, e.g., with IC₅₀ and Kᵢ values of less than 10 μM under conditions as described below in Examples 47 and 48, respectively. Some compounds of Formula (I) exhibit IC₅₀ and Kᵢ values of less than 1 μM (such as below 50 nM).

Compounds of Formula (I) can also be modified by appending appropriate functionalities to enhance selective biological properties. Such modifications are known in the art and include those that increase biological penetration into a given biological system (e.g., blood, lymphatic system, or central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

The present invention also features a pharmaceutical composition comprising a compound of Formula (I) (or a combination of two or more compounds of Formula (I)) and at least one pharmaceutically acceptable carrier. Also included in the present invention is a medicament composition including any of the compounds of Formula (I), alone or in a combination, together with a suitable excipient.

The invention also features a method of inhibiting the TGFβ family type I receptors, Alk5 and/or Alk4 (e.g., with an IC₅₀ value of less than 10 μM; such as, less than 1 μM; and for example, less than 5 nM) in a cell, including the step of contacting the cell with an effective amount of one or more compounds of Formula (I). Also within the scope of the invention is a method of inhibiting the TGFβ and/or activin signaling pathway in a cell or in a subject (e.g., a mammal such as a human), including the step of contacting the cell with or administering to the subject an effective amount of one or more of the compounds of Formula (I).

Still another aspect of this invention relates to a method of inhibiting the TGFβ signaling pathway in a subject, wherein the method includes administering to the subject in need thereof an effective amount of at least one of the compounds described above.

Still further another aspect of this invention relates to a method of inhibiting the TGFβ type I receptor in a cell, wherein the method includes contacting the cell with an effective amount of at least one of the compounds described above.

The invention further relates to a method of reducing the accumulation of excess extracellular matrix induced by TGFβ in a subject, wherein the method includes administering to the subject in need thereof an effective amount of at least one of the compounds described above.

The invention still further relates to a method of treating or preventing a fibrotic condition in a subject, wherein the method includes administering to the subject in need thereof an effective amount of at least one of the compounds described above. Examples of the fibrotic conditions subject to this method include, but are not limited to, scleroderma, lupus nephritis, connective tissue disease, wound healing, surgical scarring, spinal cord injury, CNS scarring, acute lung injury, idiopathic pulmonary fibrosis, radiation-induced pulmonary fibrosis, chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, fatty liver disease, primary sclerosing cholangitis, restenosis, radiation-induced fibrosis, chemotherapy-induced fibrosis, cardiac fibrosis, opthalnic scarring, fibrosclerosis, a fibrotic cancer, a fibroid, fibroma, a fibroadenoma, a fibrosarcoma, transplant arteriopathy, mesothelioma, and keloid.

The invention further relates to a method of inhibiting growth or metastasis of tumor cells or cancer in a subject, wherein the method includes administering to the subject in need thereof an effective amount of at least one of the compounds described above.

The invention further relates to a method of treating carcinomas mediated by an overexpression of TGFβ, wherein the method includes administering to a subject in need of such treatment an effective amount of at least one of the compounds described above. Examples of the carcinomas subject to the method include, but are not limited to, carcinomas of the lung, breast, liver, biliary tract, gastrointestinal tract, head and neck, pancreas, prostate, cervix, multiple myeloma, melanoma, glioma, and glioblastomas.

The invention further relates to a method of treating a disease or disorder mediated by an overexpression of TGFβ in a subject, wherein the method includes administering to the subject in need thereof an effective amount of at least one of the compounds described above. Examples of the diseases or disorders subject to this method include, but are not limited to, demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, head, and neck.

The invention still further relates to a method of treating or preventing restinosis, vascular disease, or hypertension, wherein the method includes administering to a subject in need thereof at least one of the compounds described above. Examples of restinosis subject to this method include, but are not limited to, coronary restenosis, peripheral restenosis, and carotid restenosis. Examples of the vascular diseases subject to this methods include, but are not limited to, intimal thickening, vascular remodeling, and organ transplant-related vascular disease. Examples of the hypertension subject to this method include, but are not limited to, primary or secondary hypertension, systolic hypertension, pulmonary hypertension, and hypertension-induced vascular remodeling. In this method, the compound can be administered locally or via an implantable device (e.g., a delivery pump or a stent).

Also within the scope of the present invention is a method of treating a subject or preventing a subject from suffering a condition characterized by or resulted from an elevated level of TGFβ and/or activin activity. The method includes the step of administering to the subject an effective amount of one or more of a compound of Formula (I). The conditions include an accumulation of excess extracellular matrix; a fibrotic condition (which can be induced by drug or radiation), e.g., scleroderma, lupus nephritis, connective tissue disease, wound healing, surgical scarring, spinal cord injury, CNS scarring, acute lung injury, pulmonary fibrosis (such as idiopathic pulmonary fibrosis and radiation-induced pulmonary fibrosis), chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, cirrhosis due to fatty liver disease (alcoholic and nonalcoholic steatosis), primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, and keloid); TGFβ-induced growth or metastasis of tumor/cancer cells; and carcinomas (e.g, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, and head and neck); and other conditions such as cachexia, hypertension, ankylosing spondylitis, demyelination in multiple sclerosis, cerebral angiopathy and Alzheimer's disease.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito, 1999, and “Advanced Organic Chemistry”, 5th Ed. (Eds.: Smith, M. B. and March, J.), John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, or heterocycloaliphaticamino); sulfonyl (e.g., aliphatic-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, some examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (such as alkyl-S(O)₂-aminoalkyl); aminoalkyl; amidoalkyl; (cycloaliphatic)alkyl; or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, heterocycloaliphaticamino, or aliphaticsulfonylamino); sulfonyl (e.g., alkyl-S(O)₂—, cycloaliphatic-S(O)₂—, or aryl-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroaralkoxy; alkoxycarbonyl; alkylcarbonyloxy; or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-S(O)₂-aminoalkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl; heteroaroyl; alkoxy; cycloalkyloxy; heterocycloalkyloxy; aryloxy; heteroaryloxy; aralkyloxy; nitro; carboxy; cyano; halo; hydroxy; sulfo; mercapto; sulfanyl (e.g., aliphatic-S- or cycloaliphatic-S—); sulfinyl (e.g., aliphatic-S(O)— or cycloaliphatic-S(O)—); sulfonyl (e.g., aliphatic-S(O)₂—, aliphaticamino-S(O)₂—, or cycloaliphatic-S(O)₂—); amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl); urea; thiourea; sulfonamide; sulfamide; alkoxycarbonyl; alkylcarbonyloxy; cycloaliphatic; heterocycloaliphatic; aryl; heteroaryl; acyl (e.g., (cycloaliphatic)carbonyl or (heterocycloaliphatic)carbonyl); amino (e.g., aliphaticamino); sulfoxy; oxo; carbamoyl; (cycloaliphatic)oxy; (heterocycloaliphatic)oxy; or (heteroaryl)alkoxy.

As used herein, an “amido” encompasses both “aminocarbonyl” and “carbonylamino.” These terms when used alone or in connection with another group refers to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl]; sulfonyl (e.g., aliphatic-S(O)₂— or amino-S(O)₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfonamide; sulfamide; or carbamoyl. Alternatively, an aryl can be unsubstituted.

Non-limiting examples of substituted aryls include haloaryl (e.g., mono-, di (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfonamide)aryl (e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl); cycloaliphatic (e.g., cycloalkyl or cycloalkenyl); (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino); cyano; halo; hydroxy; acyl; mercapto; alkylsulfanyl; sulfoxy; urea; thiourea; sulfonamide; sulfamide; oxo; or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl. A cycloalkyl or cycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic) aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); cyano; halo; hydroxy; mercapto; sulfonyl (e.g., alkyl-S(O)₂— and aryl-S(O)₂—); sulfinyl (e.g., alkyl-S(O)—); sulfanyl (e.g., alkyl-S—); sulfoxy; urea; thiourea; sulfonamide; sulfamide; oxo; or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); nitro; cyano; halo; hydroxy; mercapto; sulfonyl (e.g., alkylsulfonyl or arylsulfonyl); sulfinyl (e.g., alkylsulfinyl); sulfanyl (e.g., alkylsulfanyl); sulfoxy; urea; thiourea; sulfonamide; sulfamide; oxo; or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridinyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridinyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-S(O)₂— or amino-S(O)₂—); sulfinyl (e.g., aliphatic-S(O)—); sulfanyl) (e.g., aliphatic-S—); nitro; cyano; halo; hydroxy; mercapto; sulfoxy; urea; thiourea; sulfonamide; sulfamide; or carbamoyl. Alternatively, a heteroaryl can be unsubstituted.

Non-limiting examples of substituted heteroaryls include (halo)heteroaryl (e.g., mono- and di-(halo)heteroaryl]; (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfonamide)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; [((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl, and (haloalkyl)heteroaryl (e.g., trihaloalkylheteroaryl).

A “heteroaraliphatic (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (e.g., carboxyalkyl, hydroxyalkyl, and haloalkyl such as trifluoromethyl); alkenyl; alkynyl; cycloalkyl; (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; aminocarbonyl; alkylcarbonylamino; cycloalkylcarbonylamino; (cycloalkylalkyl)carbonylamino; arylcarbonylamino; aralkylcarbonylamino; (heterocycloalkyl)carbonylamino; (heterocycloalkylalkyl)carbonylamino; heteroarylcarbonylamino; heteroaralkylcarbonylamino; cyano; halo; hydroxy; acyl; mercapto; alkylsulfanyl; sulfoxy; urea; thiourea; sulfonamide; sulfamide; oxo; or carbamoyl.

As used herein, an “acyl” group refers to a formyl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group; or —OC(O)— or —C(O)O— when used as an internal group.

As used herein, a “haloaliphatic” group refers to an aliphatic group substituted with 1 to 3 halogen atoms. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}— S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)—when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}— CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —N(R^{X})C═(N(R^{X}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, “cyclic group” includes mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings have at least two common atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxatricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfonamide, sulfamide, oxo, or carbamoyl.

As used herein, an “aliphatic chain” refers to a branched or straight aliphatic group (e.g., alkyl groups, alkenyl groups, or alkynyl groups). A straight aliphatic chain has the structure —(CH₂)ᵥ—, where v is 1-6. A branched aliphatic chain is a straight aliphatic chain that is substituted with one or more aliphatic groups. A branched aliphatic chain has the structure —(CHQ)ᵥ— where Q is hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R¹, R², R³, R⁴, and other variables contained therein Formula (I) encompass specific groups, such as alkyl and aryl. Unless otherwise noted, each of the specific groups for the variables R¹, R², R³, R⁴, and other variables contained therein can be optionally substituted with one or more substituents described herein. Each substituent of a specific group is further optionally substituted with one to three of halo, cyano, hydroxy, amino, nitro, aryl, haloalkyl, and alkyl. For instance, an alkyl group can be substituted with alkylsulfanyl and the alkylsulfanyl can be optionally substituted with one to three of halo, cyano, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an effective amount is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

An antagonist, as used herein, is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

As compounds of Formula (I) are antagonists of TGFβ receptor type I (Alk5) and/or activin receptor type I (Alk4), these compounds are useful in inhibiting the consequences of TGFβ and/or activin signal transduction such as the production of extracellular matrix (e.g., collagen and fibronectin), the differentiation of stromal cells to myofibroblasts, and the stimulation of and migration of inflammatory cells. Thus, compounds of Formula (I) inhibit pathological inflammatory and fibrotic responses and possess the therapeutic utility of treating and/or preventing disorders or diseases for which reduction of TGFβ and/or activin activity is desirable (e.g., various types of fibrosis or progressive cancers). In addition, the compounds of Formula (I) are useful for studying and researching the role of TGFβ receptor type I (Alk5) and/or activin receptor type I (Alk4), such as their role in cellular processes, for example, signal transduction, production of extracellular matrix, the differentiation of stromal cells to myofibroblasts, and the stimulation of and migration of inflammatory cells.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### DETAILED DESCRIPTION OF THE INVENTION

In general, the invention features compounds of Formula (I), which exhibit surprisingly high affinity for the TGFβ family type I receptors, Alk 5 and/or Alk 4.

### Synthesis of the Compounds of Formula (I)

The compounds of the invention may be prepared by known methods. In general, the compounds may be prepared as illustrated in Scheme I.

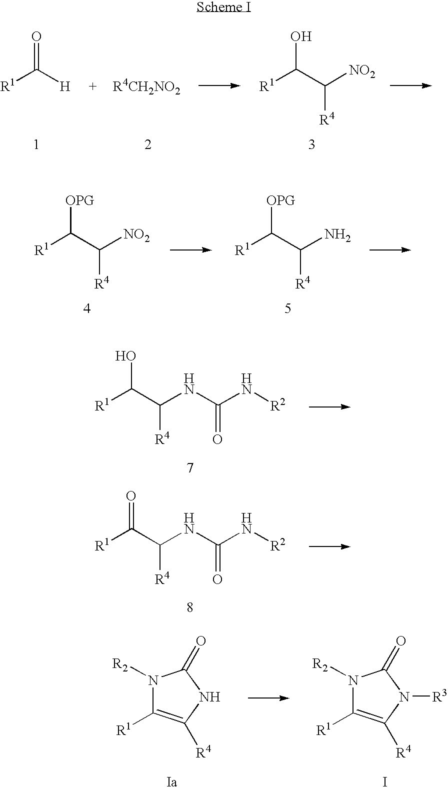

Referring to Scheme I, an aldehyde of Formula I is condensed with a nitro compound 2 to provide the nitro-alcohol 3. The condensation is performed in the presence of a base such as, for example, sodium methoxide in methanol at temperatures of from about −10° C. to about 25° C. Using known methods (see, e.g., T. W. Greene and P. G. M. Wuts, Protective Groups in Organic Synthesis, 3rd edition, John Wiley and Sons, Inc. (1999)), the alcohol of 3 is protected with a protecting group PG such as, for example, t-butyldimethylsilyl, to give the protected nitro-alcohol 4. Reduction of the nitro group of 4 to provide the protected amino-alcohol 5 may be accomplished with, for example, ammonium formate in the presence of a palladium on carbon catalyst in a protic solvent such as, for example, methanol. Reaction of the amino-alcohol 5 with an isocyanate provides the urea 6. Removal of the protecting group is performed using known conditions (ibid.) to provide the urea-alcohol 7. Oxidation of the alcohol in Formula 7 is performed with known oxidizing reagents such as, for example, Dess-Martin periodoindane to give the ketone-urea 8. Cyclization of 8 under acidic conditions such as, for example, hydrochloric acid, provides compounds of Formula (I) a (corresponding to Formula I wherein R³ is H). Further modification of compounds of Formula (I) a may be achieved by treating them with a base such as sodium hydride or potassium t-butoxide, followed by alkylation with an alkyl halide, acylation with an acyl halide or sulfonation with a sulfonyl halide.

Compounds wherein R³ is aryl or heteroaryl may be prepared as outlined in Scheme 2.

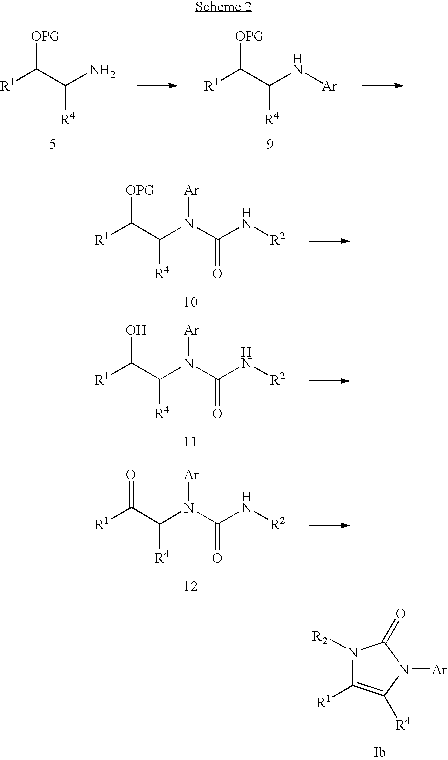

Referring to Scheme 2, the protected amino-alcohol 5 is coupled with an aryl halide following known procedures (see, e.g., http://www.organic-chemistry.org/namedreactions/buchwald-hartwig-reaction.shtm) to provide the arylated amine 9, wherein Ar means aryl or heteroaryl. Subsequent steps follow those corresponding to Scheme I to provide compounds of the invention Ib wherein R³ is aryl or heteroaryl.

### Uses of Compounds of Formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E., J. Clin. Invest. 90:1-7 (1992) and Border, W. A. and Noble, N. A. N. Engl. J. Med. 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis. Elevated TGFβ and/or activin is has also been demonstrated in cachexia, demyelination of neurons in multiple sclerosis, Alzheimer's disease, cerebral angiopathy and hypertension. Compounds of Formula (I), which are antagonists of the TGFβ family type I receptors Alk5 and/or Alk4, and inhibit TGFβ and/or activin signaling pathway, are therefore useful for treating and/or preventing fibrotic disorders or diseases mediated by an increased level of TGFβ and/or activin activity. As used herein, a compound inhibits the TGFβ family signaling pathway when it binds (e.g., with an IC₅₀ value of less than 10 μM; such as, less than 1 μM; and for example, less than 5 nM) to a receptor of the pathway (e.g., Alk5 and/or Alk4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any condition (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as scleroderma, glomerulonephritis, diabetic nephropathy, lupus nephritis, hypertension-induced nephropathy, ocular or corneal scarring, alimentary track or gastrointestinal fibrosis, renal fibrosis, hepatic or biliary fibrosis, acute lung injury, pulmonary fibrosis (such as idiopathic pulmonary fibrosis and radiation-induced pulmonary fibrosis), post-infarction cardiac fibrosis, fibrosclerosis, fibrotic cancers, fibroids, fibroma, fibroadenomas, and fibrosarcomas. Other fibrotic conditions for which preventive treatment with compounds of Formula (I) can have therapeutic utility include radiation-induced fibrosis, chemotherapy-induced fibrosis, and surgically-induced scarring including surgical adhesions, laminectomy, and coronary restenosis.

Increased TGFβ activity is also found to manifest in patients with progressive cancers. Studies have shown that in many cancers, the tumor cells, stromal cells, and/or other cells within a tumor generally overexpress TGFβ. This leads to stimulation of angiogenesis and cell motility, suppression of the immune system, and/or increased interaction of tumor cells with the extracellular matrix. See, e.g., Hojo, M. et al., Nature 397: 530-534 (1999) and Lammerts E. et al., Int. J. Cancer 102: 453-462 (2002). As a result, the tumors grow more readily, become more invasive and metastasize to distant organs. See, e.g., Maehara, Y. et al., J. Clin. Oncol. 17: 607-614 (1999) and Picon, A. et al., Cancer Epidemiol. Biomarkers Prev. 7: 497-504 (1998). Thus, compounds of Formula (I), which are antagonists of the TGFβ type I receptor and inhibit TGFβ signaling pathways, are also useful for treating and/or preventing various cancers which overexpress TGFβ or benefit from TGFβ's above-mentioned pro-tumor activities. Such cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic, and in some cases localized, nature of disorders or diseases mediated by overexpression of TGFβ and/or activin (e.g., fibrosis or cancers), small molecule treatments (such as treatment disclosed in the present invention) are favored for long-term treatment.

Not only are compounds of Formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med. 342(18): 1350-1358 (2000); Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); and Sugiyama, M. et al., Gastroenterology 114: 550-558 (1998).

### Administration of Compounds of Formula (I)

As defined above, an effective amount is the amount required to confer a therapeutic effect on the treated patient. For a compound of Formula (I), an effective amount can range, for example, from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of Formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile, such as a lizard).

Optionally, compounds of Formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid and steroid anti-inflammatory agents, immunotherapeutics, chemotherapeutics, as well as agents that antagonize ligand binding or activation of the TGFβ receptors, e.g., anti-TGFβ anti-TGFβ receptor antibodies, or antagonists of the TGFβ type II receptors. Compounds of Formula (I) can also be administered in conjunction with other treatments, e.g., radiation.

The invention will be further described in the following examples, which do not limit the scope of the invention described in the claims. Nomenclature of compounds is based on Chemdraw Ultra, version 9.0.1, Cambridgesoft.

### Preparation 1: 1-([1,2,4]triazolo[1,5-a]pyridin-6-yl)-2-nitropropan-1-ol

Into a Vial was added 0.50 M of Sodium methoxide in methanol (130 mL). This was cooled at 0° C. and nitroethane (50 mL, 0.7 mol.) was added followed by [1,2,4]triazolo[1,5-a]pyridine-6-carbaldehyde (3.35 g, 0.0228 mol.). The reaction was allowed to warm to room temperature with stirring overnight. LCMS shows conversion to a new peak consistent with the product (RT=0.51 min, m/z=223.20 M+H). The reaction was concentrated and then dissolved in EA and rinsed with 5% citric acid then dried and concentrated to give a white powder that was taken on directly into the next step. MS (ESI (+) m/z): 223.20 (M+H+)

### Preparation 2: 6-(1-(tert-butyldimethylsilyloxy)-2-nitropropyl)-[1,2,4]triazolo[1,5-a]pyridine

Into a 1-neck round-bottom flask was dissolved 2-nitro-1-[1,2,4]triazolo[1,5-a]pyridin-6-yl-propan-1-ol (5.06 g, 0.0228 mol.) in N,N-dimethylformamide (8.0 μl mL, 1.0 mol.) at 5° C. To this was added tert-butyldimethylsilyl chloride (10.5 g, 0.0699 mol.) and 1H-Imidazole (7.86 g, 0.115 mol.). The reaction was allowed to warm to room temperature, and was stirred at room temperature o/n. LCMS shows high conversion to a new peak that is consistent with the product (RT=1.74 min, m/z=337.42 M+H). The reaction was evaporated, and then dissolved in EA and washed 1× with 5% citric acid 1× with bicarb and 1× with brine before concentration and dilution in methylene chloride for purification by ISCO CombiFlash silica gel chromatography to yield 6.00 g of the title compound.

MS (ESI (+) m/z): 337.42 (M+H+).

### Preparation 3: 1-([1,2,4]triazolo[1,5-a]pyridin-6-yl)-1-(tert-butyldimethylsilyloxy)propan-2-amine

Into a 1-neck round-bottom flask was dissolved 6-[1-(tert-butyl-dimethyl-silanyloxy)-2-nitro-propyl]-[1,2,4]triazolo[1,5-a]pyridine (3.00 g, 0.00892 mol.) in Methanol (53 mL, 1.3 mol.). To this solution at 5° C. was added ammonium formate (3.01 g, 0.0478 mol.) and 5% Pd/C (5:95, palladium:carbon black, 1000 mg). The reaction was allowed to warm to room temperature and was stirred for 4 hours. LCMS analysis showed some conversion to a new peak consistent with the product (RT=1.11 min, m/z=307.28 M+H) and mostly SM. An additional gram of Pd and 3.0 grams of ammonium formate was added and the reaction finally proceeded to completion after leaving it o/n. The reaction was filtered through Celite, washed with methanol and evaporated to give the product that was brought directly on to the next step. MS (ESI (+) m/z): 307.28 (M+H+)

### Preparation 4: 1-(1-([1,2,4]triazolo[1,5-a]pyridin-6-yl)-1-(tert-butyldimethylsilyloxy)propan-2-yl)-3-m-tolylurea

Into a 1-neck round-bottom flask was added 2-(tert-butyl-dimethyl-silanyloxy)-1-methyl-2-[1,2,4]triazolo[1,5-a]pyridin-6-yl-ethylamine (1600 mg, 0.0051 mol.), 1-isocyanato-3-methyl-benzene (710 uL, 0.0056 mol.) and tetrahydrofuran (15 mL, 0.18 mol.). The reaction was stirred at room temperature for 1.5 hours. LCMS shows no starting material and two major products: RT=1.74 min (m/z=440.53 M+H) and RT=1.69 (m/z=456.34) the later peak is probably due to incomplete reduction of the nitro from the previous step. The reaction was evaporated to dryness. The residue was taken up in ethyl acetate and washed once with saturated bicarbonate before drying and concentration. The compound was then purified by isco flash chromatography hex/ea 0-100 percent after loading in methylene chloride. Pure fractions were concentrated to give 450 mgs of the title compound. MS (ESI (+) m/z): 440.53 (M+H+)

### Preparation 5: 1-(1-([1,2,4]triazolo[1,5-a]pyridin-6-yl)-1-hydroxypropan-2-yl)-3-m-tolylurea

Into a vial was added 1-[2-(tert-butyl-dimethyl-silanyloxy)-1-methyl-2-[1,2,4]triazolo[1,5-a]pyridin-6-yl-ethyl]-3-m-tolyl-urea (4330 mg, 0.00985 mol.) in ethanol (120 mL, 2.0 mol.). To this was added 12 M of hydrogen chloride in water (4.0E1 mL). A magnetic stir bar was added and the reaction was sealed and heated at 100° C. After 2 hours, LCMS shows no starting material. A new peak which was consistent with the product (RT=2.20 min, m/z=326.23 M+H) was present. The reaction was evaporated to dryness. The residue was dissolved in DMSO and purified by GILSON preparative HPLC to give the product as a white solid (TFA salt). MS (ESI (+) m/z): 326.23 (M+H+).

### Preparation 6: 1-(1-([1,2,4]triazolo[1,5-a]pyridin-6-yl)-1-oxopropan-2-yl)-3-m-tolylurea

1-(2-Hydroxy-1-methyl-2-[1,2,4]triazolo[1,5-a]pyridin-6-yl-ethyl)-3-m-tolyl-urea.C₂HO₂F₃ (4.3 g) was dissolved in methylene chloride (200 mL, 3 mol.) and Dess-Martin periodinane (6.2 g, 0.014 mol.) was added. The starting material was not very soluble so the reaction was sonicated for several minutes to try and dissolve all the starting material. After about 5-10 minutes the solution was a dark red with all the starting material dissolved. The reaction was then diluted with methylene chloride and washed twice with a saturated bicarbonate 10% sodium thiosulfate solution then washed once with brine and dried over magnesium sulfate. The aqueous layer showed no product. The concentrated crude was brought directly into the next reaction

MS (ESI (+) m/z): 324.57 (M+H+)

### Example 1

### 5-([1,2,4]triazolo[1,5-a]pyridin-6-yl)-4-methyl-1-m-tolyl-1H-imidazol-2(3H)-one

1-(1-Methyl-2-oxo-2-[1,2,4]triazolo[1,5-a]pyridin-6-yl-ethyl)-3-m-tolyl-urea (3.2 g, 0.0099 mol.) crude from the previous reaction was dissolved in hydrogen chloride (100 mL, 4 mol.) and stirred at room temperature for 2 hours. LC-MS showed product formation at 2.12/306.20 and disappearance of starting material. The reaction was concentrated and diluted with DMSO and purified by preparative HPLC. The pure fractions were combined to give 885 mg of pure product as a triethylamine salt.

¹H NMR (300 MHz, DMSO-d6) δ ppm: 10.61, s, 1H, 8.86, s, 1H, 8.49, s, 1H, 7.67, d, J=10 Hz, 1H, 7.28-6.79, m, 5H, 2.24, s, 3H, 2.09, s, 3H. MS (ESI (+) m/z): 306.20 (M+H+)

### Example 2

### 5-([1,2,4]triazolo[1,5-a]pyridin-6-yl)-1-m-tolyl-1H-imidazol-2(3H)-one

Title compound was prepared as per 4-methyl-1-m-tolyl-5-[1,2,4]triazolo[1,5-a]pyridin-6-yl-1,3-dihydro-imidazol-2-one, substituting nitromethane in place of nitroethane in the first step.

¹H NMR (300 MHz, DMSO-d6) δ ppm: 10.672 (s. 1H) 8.543 (m, 1H) 8.444 (s, 1H) 7.699 (dd, J=9.3, 0.8 Hz, 1H) 7.289-7.224 (m, 2H) 7.173-7.121 (m, 2H) 7.061 (d, J=2.6 Hz, 1H) 6.949 (d, J=7.8 Hz, 1H) 2.280 (s, 3H). MS m/z=292.20 (M+1).

### Example 3

### 4-([1,2,4]triazolo[1,5-a]pyridin-6-yl)-1-(4-methoxybenzyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (16 mg, 0.000052 mol.) was dissolved in N,N-dimethylformamide (1 mL, 0.01 mol.) and cooled to 0° C. Potassium tert-butoxide (0.015 g, 0.00013 mol.) was added then p-methoxybenzyl chloride (11 uL, 0.000079 mol.). The reaction was allowed to warm to room temperature and stir for 3-4 hours. LC-MS showed formation of product at 3.02/426.30. The product was isolated by HPLC.

¹H NMR (300 MHz, DMSO-d6) δ ppm: 8.86, s, 1H, 8.49, s, 1H, 7.67, d, J=10 Hz, 1H, 7.26-6.86, m, 9H, 4.84, s, 2H, 3.74, s, 3H, 2.24, s, 3H, 2.09, s, 3H. (ESI (+) m/z): 426.30 (M+H+)

### Example 4

### 4-([1,2,4]triazolo[1,5-a]pyridin-6-yl)-1-benzyl-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50 mg, 0.0002 mol.) was dissolved in N,N-dimethylformamide (3 mL, 0.04 mol.) and cooled to 0° C. Potassium tert-butoxide (0.046 g, 0.00041 mol.) was added then Benzyl chloride (28 uL, 0.00024 mol.). The reaction was allowed to warm to room temperature and stir for 3-4 hours. LC-MS showed formation of product at 1.44/396.30. The product was isolated by Gilson preparative HPLC.

¹H NMR (300 MHz, DMSO-d6) δ ppm: 8.86, s, 1H, 8.49, s, 1H, 7.67, d, J=10 Hz, 1H, 7.44-7.28, m, 5H, 7.23-7.14, m, 3H, 7.07, d, J=8 Hz, 1H, 6.92, d, J=8 Hz, 1H, 4.96, s, 2H, 2.24, s, 3H, 2.09, s, 3H. (ESI (+) m/z): 396.30 (M+H+)

### Example 5

### Methyl 4-((4-([1,2,4]triazolo[1,5-a]pyridin-6-yl)-5-methyl-2-oxo-3-m-tolyl-2,3-dihydro-1H-imidazol-1-yl)methyl)benzoate

4-Methyl-1-m-tolyl-5-[1,2,4]triazolo[1,5-a]pyridin-6-yl-1,3-dihydro-imidazol-2-one (50 mg, 0.0002 mol.) was dissolved in N,N-dimethylformamide (3 mL, 0.04 mol.) and cooled to 0° C. Potassium tert-butoxide (0.040 g, 0.00036 mol.) was added then methyl 4-chloromethylbenzoate (0.036 g, 0.00020 mol.). The reaction was allowed to warm to room temperature and stir for 3-4 hours. LC-MS showed formation of product at 1.45/454.32. The product was isolated by Gilson preparative HPLC.

¹H NMR (300 MHz, DMSO-d6) δ ppm: 8.86, s, 1H, 8.49, s, 1H, 8.02-7.97, m, 2H, 7.67, d, J=10 Hz, 1H, 7.53-7.48, m, 2H, 7.23-6.9, m, 5H, 5.06, s, 2H, 3.86, s, 3H, 2.24, s, 3H, 2.09, s, 3H. (ESI (+) m/z): 454.32 (M+H+).

### Example 6

### 4-([1,2,4]triazolo[1,5-a]pyridin-6-yl)-5-methyl-1-(3-nitrobenzyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50 mg, 0.0002 mol.) was dissolved in N,N-dimethylformamide (3 mL, 0.04 mol.) and cooled to 0° C. Potassium tert-butoxide (0.046 g, 0.00041 mol.) was added then 1-(chloromethyl)-3-nitrobenzene (0.042 g, 0.00024 mol.). The reaction was allowed to warm to room temperature and stir for 3-4 hours. LC-MS showed formation of product at 1.45/441.47. The product was isolated by HPLC. Some starting material remained after purification therefore an isco column was run using CH₂Cl₂/CH₃OH, 0-8 percent gradient, to give the pure product.

¹H NMR (300 MHz, DMSO-d6) δ ppm: 8.95, m, 1H, 8.49, s, 1H, 8.27, m 1H, 8.22-8.17, m, 1H, 7.86-7.80, m, 1H, 7.75-7.66, m, 2H, 7.23-7.14, m, 3H, 7.11-7.06, m, 1H, 6.95-6.90, m, 1H, 5.13, s, 2H, 2.24, s, 3H, 2.09, s, 3H. (ESI (+) m/z): 441.47 (M+H+)

### Example 7

### Methyl 3-((4-([1,2,4]triazolo[1,5-a]pyridin-6-yl)-5-methyl-2-oxo-3-m-tolyl-2,3-dihydro-1H-imidazol-1-yl)methyl)benzoate

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.042 g, 0.00038 mol.) was added then 3-Chloromethyl-benzoic acid methyl ester (0.030 g, 0.00016 mol.). The reaction was allowed to warm to room temperature and stir for 3-4 hours. LC-MS showed formation of product at 1.44/454.40. The product was isolated by Gilson preparative HPLC.

¹H NMR (300 MHz, DMSO-d6) δ ppm: 8.93, s, 1H, 8.49, s, 1H; 8.01, m, 1H, 7.94-7.90, m, 1H, 7.70-7.53, m, 3H, 7.23-7.14, m, 3H, 7.10-7.05, m, 1H, 6.94-6.90, m, 1H, 5.06, s, 2H, 3.86, s, 3H, 2.24, s, 3H, 2.09, s, 3H. (ESI (+) m/z): 454.40 (M+H+)

### Example 8

### 3-((4-([1,2,4]triazolo[1,5-a]pyridin-6-yl)-5-methyl-2-oxo-3-m-tolyl-2,3-dihydro-1H-imidazol-1-yl)methyl)benzamide

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.031 g, 0.00028 mol.) was added then 3-chloromethyl-benzamide (0.021 g, 0.00012 mol.). The reaction was allowed to warm to room temperature and stir for 3-4 hours. LC-MS showed formation of product at 1.13 439.28. The product was isolated by Gilson preparative HPLC.

¹H NMR (300 MHz, DMSO-d6) δ ppm: 8.93, s, 1H, 8.49, s, 1H, 8.01, m, 1H, 7.89, s, 1H, 7.81, m, 1H, 7.68, dd, J=8 Hz, 1H, 7.53-7.38, m, 3H, 7.22-7.15, m, 3H, 7.09-7.05, m, 1H, 6.94-6.89, m, 1H, 5.01, s, 2H, 2.24, s, 3H, 2.09, s, 3H. (ESI (+) m/z): 439.28 (M+H+)

### Example 9

### 4-([1,2,4]triazolo[1,5-a]pyridin-6-yl)-1,5-dimethyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.042 g, 0.00038 mol.) was added then methyl iodide (0.011 mL, 0.00018 mol.). The reaction was allowed to warm to room temperature and stir for 3-4 hours. LC-MS showed formation of product at 1.08 320.52. The product was isolated by Gilson Preparative HPLC.

¹H NMR (300 MHz, DMSO-d6): 6 ppm: 8.93, s, 1H, 8.49, s, 1H, 7.68, dd, J=8 Hz, 1H, 7.21-7.02, m, 4H, 6.89-6.84, m, 1H, 3.25, s, 3H, 2.24, s, 3H, 2.09, s, 3H. (ESI (+) m/z): 320.52 (M+H+).

### Example 10

### 4-([1,2,4]triazolo[1,5-a]pyridin-6-yl)-1-(cyclohexylmethyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.042 g, 0.00038 mol.) was added then bromomethylcyclohexane (27 uL, 0.00020 mol.). The reaction was allowed to warm to room temperature and stir for 3-4 hours. Potassium iodide (0.054 g, 0.00033 mol.) was added. LC-MS showed formation of product at 1.64/402.83 and disappearance of starting material. The product was isolated by Gilson preparative HPLC.

¹H NMR (300 MHz, DMSO-d6) δ ppm: 8.93, s, 1H, 8.49, s, 1H, 7.68, dd, J=8 Hz, 1H, 7.21-7.02, m, 4H, 6.89-6.84, m, 1H, 3.52, d, J=7 Hz, 2H, 2.24, s, 3H, 2.09, s, 3H, 1.81-1.59, m, 6H, 1.28-1.11, m, 3H, 1.10-0.95, m, 2H. (ESI (+) m/z): 402.83 (M+H+)

### Example 11

### 4-([1,2,4]triazolo[1,5-a]pyridin-6-yl)-1-(3-aminobenzyl)-5-methyl-3-m-tolyl-1H-imidazol-2(3H)-one

4-Methyl-3-(3-nitrobenzyl)-1-m-tolyl-5-[1,2,4]triazolo[1,5-a]pyridin-6-yl-1,3-dihydroimidazol-2-one (0.025 g, 0.000057 mol.) was dissolved in methanol (3 mL, 0.07 mol.) and 5% Pd/C (5:95, palladium:carbon black, 0.010 g) was added then ammonium formate (0.07 g, 0.001 mol.). The reaction was sealed and allowed to stir at room temperature for 2 hours. LC-MS showed product formation at 0.98/411.66. The mixture was filtered through a 0.45 uM filter and then purified by Gilson preparative HPLC to give the pure product. (ESI (+) m/z): 411.66 (M+H+).

### Example 12

### 4-([1,2,4]triazolo[1,5-a]pyridin-6-yl)-5-methyl-1-(pyridin-2-ylmethyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.018 g, 0.00016 mol.) was added then 2-bromomethyl-pyridine.HBr (0.050 g). The reaction was allowed to warm to room temperature and stir for 3-4 hours. LC-MS showed formation of product at 0.95/397.67. An additional 1 eq of potassium t-butoxide was added and the reaction was allowed to stir for several hours. After stirring overnight the product was isolated by Gilson preparative HPLC.

¹H NMR (300 MHz, THF-d8): 8.70, m, 1H, 8.52, s, 1H, 8.22, s 1H, 7.74-7.67, m, 1H, 7.52-7.39, m, 2H, 7.24-6.94, m, 6H, 6.89-6.84, m, 1H, 5.04, s, 2H, 2.25, s, 3H, 2.23, s, 3H. (ESI (+) m/z): 397.67 (M+H+).

### Example 13

### 4-([1,2,4]triazolo[1,5-a]pyridin-6-yl)-5-methyl-1-(pyridin-3-ylmethyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.064 g, 0.00057 mol.) was added then 3-bromomethyl-pyridine.HBr (0.050 g). The reaction was allowed to warm to room temperature and stir for 3-4 hours. LC-MS showed formation of product at 0.91/397.51. An additional 1 eq of base was added and the mixture stirred for several hours. The product was isolated by Gilson preparative HPLC.

¹H NMR (300 MHz, DMSO-d6) 8 ppm: 8.94, m, 1H, 8.73, m, 1H, 8.65, m, 1H, 8.52, s, 1H, 8.00, m, 1H, 7.72-7.59, m, 2H, 7.22-7.05, m, 4H, 6.92, m, 1H, 5.07, s, 2H, 2.25, s, 3H, 2.23, s, 3H. (ESI (+) m/z): 397.51 (M+H+)

### Example 14

### 4-([1,2,4]triazolo[1,5-a]pyridin-6-yl)-1-(2-methoxyethyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.042 g, 0.00038 mol.) was added followed by 1-bromo-2-methoxy ethane (0.2 uL, 0.00021 mol.) and potassium iodide (0.054 g, 0.00033 mol). LC-MS after stirring overnight showed formation of product at 1.14/363.99. The product was isolated by Gilson Preparative HPLC.

¹H NMR (300 MHz, DMSO-d6) δ ppm: 8.93, s, 1H, 8.49, s, 1H, 7.68, d, J=8 Hz, 1H, 7.21-7.02, m, 4H, 6.89-6.84, m, 1H, 3.85, t, J=6 Hz, 2H, 3.58, t, J=6 Hz, 2H, 3.31, s, 3H, 2.24, s, 3H, 2.09, s, 3H. (ESI (+) m/z): 363.99 (M+H+)

### Example 15

### Ethyl 2-(4-([1,2,4]triazolo[1,5-a]pyridin-6-yl)-5-methyl-2-oxo-3-m-tolyl-2,3-dihydro-1H-imidazol-1-yl)acetate

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then ethyl bromoacetate (24 uL, 0.00021 mol.). The reaction was allowed to warm to room temperature and stir for 3-4 hours. LC-MS showed formation of product at 1.23/392.29. The product was isolated by Gilson preparative HPLC.

¹H NMR (300 MHz, DMSO-d6) δ ppm: 8.93, s, 1H, 8.49, s, 1H, 7.68, d, J=8 Hz, 1H, 7.21-7.02, m, 4H, 6.89-6.84, m, 1H, 4.61, s, 2H, 4.21, q, J=7 Hz, 2H, 2.24, s, 3H, 2.09, s, 3H, 1.26, t, J=6 Hz, 3H. (ESI (+) m/z): 392.29 (M+H+)

### Example 16

### 4-([1,2,4]triazolo[1,5-a]pyridin-6-yl)-1-(benzo[c][1,2,5]oxadiazol-5-ylmethyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 5-bromomethyl-benzo[c][1,2,5]oxadiazole (0.038 g, 0.00018 mol.). The reaction was allowed to warm to room temperature and stir for 3-4 hours. LC-MS showed formation of product at 1.45/438.27. The product was isolated by Gilson Preparative HPLC.

¹H NMR (300 MHz, DMSO-d6) δ ppm: 8.94, m, 1H, 8.52, s, 1H, 8.12, m, 1H, 7.90, s, 1H, 7.72-7.59, m, 2H, 7.25-7.16, m, 3H, 7.09, m, 1H, 6.95, m, 1H, 5.13, s, 2H, 2.25, s, 3H, 2.15, s, 3H. (ESI (+) m/z): 438.27 (M+H+)

### Example 17

### 4-([1,2,4]triazolo[1,5-a]pyridin-6-yl)-5-methyl-1-((5-methylisoxazol-3-yl)methyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 3-bromomethyl-5-methyl-isoxazole (0.032 g, 0.00018 mol.). The reaction was allowed to warm to room temperature and stir for 3-4 hours. LC-MS showed formation of product at 1.25/401.31. The product was isolated by Gilson preparative HPLC.

¹H NMR (300 MHz, DMSO-d6) δ ppm: 8.94, m, 1H, 8.52, s, 1H, 7.69, d, J=6 Hz, 1H, 7.22-7.04, m, 4H, 6.88, m, 1H, 6.26, s, 1H, 4.97, s, 2H, 2.41, s, 3H, 2.25, s, 3H, 2.15, s, 3H. (ESI (+) m/z): 401.31 (M+H+)

### Example 18

### 4-([1,2,4]triazolo[1,5-a]pyridin-6-yl)-5-methyl-1-((tetrahydro-2H-pyran-4-yl)methyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then sodium iodide (0.061 g, 0.00041 mol.) and finally 4-bromomethyltetrahydropyran (0.038 g, 0.00021 mol). The reaction was then heated at 50° C. for 5 hours. LC-MS showed formation of product at 1.13/404.37 which was isolated by Gilson preparative HPLC.

¹H NMR (300 MHz, DMSO-d6) δ ppm: 8.93, s, 1H, 8.49, s, 1H, 7.68, d, J=8 Hz, 1H, 7.21-7.02, m, 4H, 6.89-6.84, m, 1H, 3.88, m, 2H, 3.58, m, 2H, 3.29, t, J=12 Hz, 2H, 2.24, s, 3H, 2.19, s, 3H, 2.00, m, 1H, 1.58, m, 2H, 1.31, m, 2H. (ESI (+) m/z): 404.37 (M+H+)

### Example 19

### 3-((4-([1,2,4]triazolo[1,5-a]pyridin-6-yl)-5-methyl-2-oxo-3-m-tolyl-2,3-dihydro-1H-imidazol-1-yl)methyl)benzonitrile

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then m-cyanobenzyl bromide (0.035 g, 0.00018 mol.). The reaction was allowed to warm to room temperature and stir for 3-4 hours. LC-MS showed formation of product at 1.39/421.37. The product was isolated by Gilson preparative HPLC.

¹H NMR (300 MHz, DMSO-d6) δ ppm: 8.95, m, 1H, 8.49, s, 1H, 7.86-7.78, m 2H, 7.74-7.59, m, 3H, 7.23-7.14, m, 3H, 7.08, m, 1H, 6.92, m, 1H, 5.03, s, 2H, 2.24, s, 3H, 2.09, s, 3H. (ESI (+) m/z): 421.37 (M+H+)

### Example 20

### 4-([1,2,4]triazolo[1,5-a]pyridin-6-yl)-1-(3-fluorobenzyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformarnmide (3 mL, 0.04 mol.) and cooled to 0° C. Potassium tert-butoxide (0.046 g, 0.00041 mol.) was added then α-bromo-3-fluorotoluene (22 uL, 0.00018 mol.). The reaction was allowed to warm to room temperature and stir for 3-4 hours. LC-MS showed formation of product at 1.49/414.31. The product was isolated by Gilson preparative HPLC.

¹H NMR (300 MHz, DMSO-d6) δ ppm: 8.95, m, 1H, 8.49, s, 1H, 7.69, m 1H, 7.45, m, 1H, 7.25-7.05, m, 6H, 6.92, m, 1H, 4.99, s, 2H, 2.24, s, 3H, 2.09, s, 3H. (ESI (+) m/z): 414.31 (M+H+)

### Example 21

### 4-([1,2,4]triazolo[1,5-a]pyridin-6-yl)-5-methyl-1-(3-methylbenzyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 1-bromomethyl-3-methylbenzene (24 uL, 0.00018 mol.). The reaction was allowed to warm to room temperature and stir for 3-4 hours. LC-MS showed formation of product at 1.56/410.49. The product was isolated by Gilson preparative HPLC.

¹H NMR (300 MHz, DMSO-d6) δ ppm: 8.95, m, 1H, 8.49, s, 1H, 7.69, m 1H, 7.32-7.05, m, 8H, 6.92, m, 1H, 4.92, s, 2H, 2.32, s, 3H, 2.24, s, 3H, 2.09, s, 3H. (ESI (+) m/z): 410.49 (M+H+)

### Example 22

### 4-([1,2,4]triazolo[1,5-a]pyridin-6-yl)-1-(3-methoxybenzyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 3-methoxybenzyl bromide (25 uL, 0.00018 mol.). The reaction was allowed to warm to room temperature and stir for 3-4 hours. LC-MS showed formation of product at 1.47/426.77. The product was isolated by Gilson preparative HPLC.

¹H NMR (300 MHz, DMSO-d6) δ ppm: 8.95, m, 1H, 8.49, s, 1H, 7.69, m 1H, 7.31, t, J=8 Hz, 1H, 7.23-7.13, m, 3H, 7.07, m, 1H, 6.95-6.85, m, 4H, 4.92, s, 2H, 3.76, s, 3H, 2.24, s, 3H, 2.09, s, 3H. (ESI (+) m/z): 426.77 (M+H+)

### Example 23

### 2-((4-([1,2,4]triazolo[1,5-a]pyridin-6-yl)-5-methyl-2-oxo-3-m-tolyl-2,3-dihydro-1H-imidazol-1-yl)methyl)benzonitrile

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 2-(bromomethyl)benzonitrile (0.035 g, 0.00018 mol.). The reaction was allowed to warm to room temperature and stir for 3-4 hours. LC-MS showed formation of product at 1.39/421.41. The product was isolated by Gilson preparative HPLC.

¹H NMR (300 MHz, DMSO-d6) δ ppm: 8.95, m, 1H, 8.49, s, 1H, 7.92, m, 1H, 7.80-7.69, m, 2H, 7.54, t, J=8 Hz, 1H, 7.45, m, 1H, 7.23-7.13, m, 3H, 7.07, m, 1H, 6.97, m, 1H, 4.92, s, 2H, 2.24, s, 3H, 2.09, s, 3H. (ESI (+) m/z): 421.41 (M+H+)

### Example 24

### 4-([1,2,4]triazolo[1,5-a]pyridin-6-yl)-5-methyl-1-(2-nitrobenzyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then o-nitrobenzylbromide (0.039 g, 0.00018 mol.). The reaction was allowed to warm to room temperature and stirred for 3-4 hours. LC-MS showed formation of product at 1.46/441.27. The product was isolated by Gilson preparative HPLC.

¹H NMR (300 MHz, DMSO-d6) δ ppm: 8.95, m, 1H, 8.49, s, 1H, 8.19, m, 1H, 7.81, t, J=8 Hz, 1H, 7.72, d, J=8 Hz, 1H, 7.62, t, J=8 Hz, 1H, 7.29-7.16, m, 4H, 7.07, m, 1H; 6.97, m, 1H, 5.33, s, 2H, 2.24, s, 3H, 2.09, s, 3H. (ESI (+) m/z): 441.27 (M+H+)

### Example 25

### 4-([1,2,4]triazolo[1,5-a]pyridin-6-yl)-5-methyl-1-(2-methylbenzyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 1-(bromomethyl)-2-methylbenzene (24 uL, 0.00018 mol.). The reaction was allowed to warm to room temperature and stirred for 3-4 hours. LC-MS showed formation of product at 1.55/410.19. The product was isolated by Gilson preparative HPLC.

¹H NMR (300 MHz, DMSO-d6) δ ppm: 8.95, m, 1H, 8.49, s, 1H, 7.71, d, J=8 Hz, 1H, 7.26-7.15, m, 6H, 7.07, m, 1H, 7.00, m, 1H, 6.93, m, 1H, 4.92, s, 2H, 2.38, s, 3H, 2.24, s, 3H, 2.09, s, 3H. (ESI (+) m/z): 410.19 (M+H+)

### Example 26

### 4-([1,2,4]triazolo[1,5-a]pyridin-6-yl)-1-(benzo[c][1,2,5]thiadiazol-5-ylmethyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then 5-(bromomethyl)benzo[c][1,2,5]thiadiazole (0.041 g, 0.00018 mol.). The reaction was allowed to warm to room temperature and stirred for 3-4 hours. LC-MS showed formation of product at 1.43/454.19. The product was isolated by Gilson preparative HPLC.

¹H NMR (300 MHz, DMSO-d6) δ ppm: 8.95, m, 1H, 8.67, m, 1H, 8.51-8.44, m, 2H, 7.86, m, 1H, 7.69, d, J=8 Hz, 1H, 7.24-7.17, m, 3H, 7.07, m, 1H, 6.93, m, 1H, 5.25, s, 2H, 2.24, s, 3H, 2.09, s, 3H. (ESI (+) m/z): 454.19 (M+H+)

### Example 27

### 4-([1,2,4]triazolo[1,5-a]pyridin-6-yl)-1-(2-aminobenzyl)-5-methyl-3-m-tolyl-1H-imidazol-2(3H)-one

4-([1,2,4]triazolo[1,5-a]pyridin-6-yl)-5-methyl-1-(2-nitrobenzyl)-3-m-tolyl-1H-imidazol-2(3H)-one (0.025 g, 0.000057 mol.) was dissolved in methanol (3 mL, 0.07 mol.) and 5% Pd/C (0.010 g) was added then ammonium formate (0.07 g, 0.001 mol.). The reaction was sealed and allowed to stir at room temperature for 2 hours. LC-MS showed product formation at 1.09/411.43. The mixture was filtered through a 0.45 uM filter and then purified by Gilson preparative HPLC to give the product. (ESI (+) m/z): 411.43 (M+H+)

### Example 28

### 4-([1,2,4]triazolo[1,5-a]pyridin-6-yl)-1-(cyclobutylmethyl)-5-methyl-3-phenyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (3 mL, 0.04 mol.) and cooled to 0° C. Potassium tert-butoxide (0.046 g, 0.00041 mol.) was added then sodium iodide (0.061 g, 0.00041 mol.) and finally bromomethylcyclobutane (24 uL, 0.00021 mol.) was added. The reaction was then heated at 80 for 5 hours. LC-MS showed product 1.45/374.20 which was isolated by Gilson preparative HPLC.

¹H NMR (300 MHz, DMSO-d6) δ ppm: 8.95, m, 1H, 8.49, s, 1H, 7.69, d, J=8 Hz, 1H, 7.21-7.02, m, 4H, 6.93, m, 1H, 3.74, d, J=8 Hz, 2H, 2.69, m, 1H, 2.24, s, 3H, 2.09, s, 3H, 2.07-1.96, m, 2H, 1.89-1.79, m, 4H. (ESI (+) m/z): 374.20 (M+H+)

### Example 29

### 4-([1,2,4]triazolo[1,5-a]pyridin-6-yl)-5-methyl-1-(1-oxo-2-oxaspiro[4.5]decan-8-yl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (50.0 mg, 0.000164 mol.) was dissolved in N,N-dimethylformamide (2 mL, 0.02 mol.) and cooled to 0° C. Potassium tert-butoxide (0.046 g, 0.00041 mol.) was added then and finally toluene-4-sulfonic acid, 1-oxo-2-oxa-spiro[4.5]dec-8-yl ester (0.372 g, 0.00115 mol.) was added. The reaction was then heated at 80 for 5 hours. Two peaks with the correct mass were found one at 1.16 preliminarily assigned as 0 alkylation and one at 1.31 preliminarily assigned as N alkylation. (ESI (+) m/z): 458.62 (M+H+)

### Example 30

### 5-([1,2,4]triazolo[1,5-a]pyridin-6-yl)-1-(3-chloro-4-fluorophenyl)-4-methyl-1H-imidazol-2(3H)-one

Synthesis was performed as described for 4-methyl-1-m-tolyl-5-[1,2,4]triazolo[1,5-a]pyridin-6-yl-1,3-dihydro-imidazol-2-one substituting 2-chloro-1-fluoro-4-isocyanato-benzene for 1-isocyanato-3-methyl-benzene step 4.

¹H NMR (300 MHz, DMSO-d6) δ ppm: 10.74, s, 1H, 8.93, s, 1H, 8.51, s, 1H, 7.72, d, J=10 Hz, 1H, 7.57, m, 1H, 7.33, t, J=10 Hz, 1H, 7.19, m, 1H, 7.05, m, 1H, 2.09, s, 3H. (ESI (+) m/z): 344.06 (M+H+)

### Example 31

### 3-((4-([1,2,4]triazolo[1,5-a]pyridin-6-yl)-5-methyl-2-oxo-3-m-tolyl-2,3-dihydro-1H-imidazol-1-yl)methyl)benzoic acid

3-(5-Methyl-2-oxo-3-m-tolyl-4-[1,2,4]triazolo[1,5-a]pyridin-6-yl-2,3-dihydro-imidazol-1-ylmethyl)-benzoic acid methyl ester (0.010 g, 0.000022 mol.) was dissolved in tetrahydrofuran (2 mL, 0.02 mol.) and 1 M lithium hydroxide in water (0.200 mL) was added. The reaction was allowed to stir overnight at room temperature and LC/MS showed product at 1.25/440.17. The reaction was concentrated, dissolved in DMSO and purified by Gilson preparative HPLC.

¹H NMR (300 MHz, DMSO-d6) δ ppm: 8.95, m, 1H, 8.49, s, 1H, 7.97, m, 2H, 7.69, d, J=8 Hz, 1H, 7.48, m, 2H, 7.23-7.15, m, 3H, 7.07, m, 1H, 6.93, m, 1H, 5.04, s, 2H, 2.24, s, 3H, 2.09, s, 3H. (ESI (+) m/z): 440.17 (M+H+)

### Example 32

### 4-((4-([1,2,4]triazolo[1,5-a]pyridin-6-yl)-5-methyl-2-oxo-3-m-tolyl-2,3-dihydro-1H-imidazol-1-yl)methyl)benzoic acid

4-(5-Methyl-2-oxo-3-m-tolyl-4-[1,2,4]triazolo[1,5-a]pyridin-6-yl-2,3-dihydro-imidazol-1-ylmethyl)-benzoic acid methyl ester (0.010 g, 0.000022 mol.) was dissolved in tetrahydrofuran (2 mL, 0.02 mol.) and 1 M lithium hydroxide in water (0.200 mL) was added. The reaction was allowed to stir overnight at room temperature. The reaction was concentrated and the residue dissolved in DMSO and purified by Gilson preparative HPLC.

¹H NMR (300 MHz, DMSO-d6) δ ppm: 8.95, m, 1H, 8.49, s, 1H, 7.99, m, 1H, 7.89, m, 1H, 7.70-7.50, m, 3H, 7.23-7.15, m, 3H, 7.07, m, 1H, 6.93, m, 1H, 5.04, s, 2H, 2.24, s, 3H, 2.09, s, 3H. (ESI (+) m/z): 440.19 (M+H+)

### Example 33

### 4-([1,2,4]triazolo[1,5-a]pyridin-6-yl)-3-(3-chloro-4-fluorophenyl)-5-methyl-1-(3-methylbenzyl)-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50.0 mg, 0.000145 mol.) was dissolved in N,N-dimethylformamide (3 mL, 0.04 mol.) and cooled to 0° C. Potassium tert-butoxide (0.023 g, 0.00020 mol.) was added then 1-bromomethyl-3-methylbenzene (0.030 g, 0.00016 mol.). The reaction was allowed to warm to room temperature and stir for 3-4 hours. LC-MS showed formation of product at 1.66/448.23. The product was isolated by Gilson preparative HPLC.

¹H NMR (300 MHz, DMSO-d6) δ ppm: 8.98, m, 1H, 8.52, s, 1H, 7.73, d, J=10 Hz, 1H, 7.65, m, 1H, 7.70-7.50, m, 3H, 7.40-7.08, m, 7H, 4.92, s, 2H, 2.32, s, 3H, 2.09, s, 3H. (ESI (+) m/z): 448.23 (M+H+)

### Example 34

### 4-([1,2,4]triazolo[1,5-a]pyridin-6-yl)-1-(benzo[c][1,2,5]oxadiazol-5-ylmethyl)-3-(3-chloro-4-fluorophenyl)-5-methyl-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50.0 mg, 0.000145 mol.) was dissolved in N,N-dimethylformamide (3 mL, 0.04 mol.) and cooled to 0° C. Potassium tert-butoxide (0.023 g, 0.00020 mol.) was added then 5-bromomethyl-benzo[c][1,2,5]oxadiazole (0.034 g, 0.00016 mol.). The reaction was allowed to warm to room temperature and stir for 3-4 hours. LC-MS showed formation of product at 1.54/476.04. The product was isolated by Gilson preparative HPLC.

¹H NMR (300 MHz, DMSO-d6) δ ppm: 9.03, m, 1H, 8.52, s, 1H, 8.13, d, J=10 Hz, 1H, 7.91, m, 1H, 7.78-7.60, m, 3H, 7.38, t, J=9 Hz, 1H, 7.28, m, 1H, 7.18, m, 1H, 5.13, s, 2H, 2.14, s, 3H. (ESI (+) m/z): 476.04 (M+H+)

### Example 35

### 4-([1,2,4]triazolo[1,5-a]pyridin-6-yl)-1-(benzo[c][1,2,5]thiadiazol-5-ylmethyl)-3-(3-chloro-4-fluorophenyl)-5-methyl-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (54.6 mg, 0.000159 mol.) was dissolved in N,N-dimethylformamide (3 mL, 0.04 mol.) and cooled to 0° C. Potassium tert-butoxide (0.025 g, 0.00022 mol.) was added then 5-(bromomethyl)benzo[c][1,2,5]thiadiazole (0.040 g, 0.00017 mol.). The reaction was allowed to warm to room temperature and stir for 3-4 hours. LC-MS showed formation of product at 1.52/492.32. The product was isolated by Gilson preparative HPLC.

¹H NMR (300 MHz, DMSO-d6) δ ppm: 9.01, m, 1H, 8.69, m, 1H, 8.52, s, 1H, 8.46, d, J=10 Hz, 1H, 7.86, m, 1H, 7.78-7.68, m, 2H, 7.38, t, J=9 Hz, 1H, 7.28, m, 1H, 7.18, m, 1H; 5.26, s, 2H, 2.16, s, 3H. (ESI (+) m/z): 492.32 (M+H+)

### Example 36

### 4-([1,2,4]triazolo[1,5-a]pyridin-6-yl)-3-(3-chloro-4-fluorophenyl)-1-(3-fluorobenzyl)-5-methyl-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50.0 mg, 0.000145 mol.) was dissolved in N,N-dimethylformamide (3 mL, 0.04 mol.) and cooled to 0° C. Potassium tert-butoxide (0.023 g, 0.00020 mol.) was added then α-bromo-3-fluorotoluene (0.030 g, 0.00016 mol.). The reaction was allowed to warm to room temperature and stir for 3-4 hours. LC-MS showed formation of product at 1.58/452.16. The product was isolated by Gilson preparative HPLC.

¹H NMR (300 MHz, DMSO-d6) δ ppm: 9.01, m, 1H, 8.69, m, 1H, 8.52, s, 1H, 7.74, d, J=10 Hz, 1H, 7.66, m, 1H, 7.50-7.30, m, 2H, 7.28-7.10, m, 5H, 4.99, s, 2H, 2.10, s, 3H. (ESI (+) m/z): 452.16 (M+H+)

### Example 37

### 4-([1,2,4]triazolo[1,5-a]pyridin-6-yl)-5-methyl-1-(4-methylbenzyl)-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (44 mg, 0.14 mmol) was dissolved in N,N-dimethylformamide (3 mL, Acros) and cooled to 0° C. Potassium tert-butoxide (26 mg, 0.23 mmol, Aldrich) was added followed by 1-bromomethyl-4-methylbenzene (39 mg, 0.21 mmol, Acros). The reaction was allowed to warm to room temperature and stir for 3 hours. The product was isolated by preparative HPLC to give the title compound as a trifluoroacetic acid salt (46.9 mg, 64%).

¹H NMR (500 MHz, DMSO-d6) δ ppm: 8.922 (s, 1H) 8.490 (s, 1H) 7.669 (d, J=9.6 Hz, 1H)

7.253 (d, J=7.9 Hz, 2H) 7.207-7.123 (m, 5H) 7.058 (d, J=7.7 Hz, 1H) 6.884 (d, J=7.9 Hz, 1H) 4.896 (s, 2H) 2.286 (s, 3H) 2.239 (s, 3H) 2.088 (s, 3H). (ESI (+) m/z): 410.35 (M+1).

### Example 38

### 4-([1,2,4]triazolo[1,5-a]pyridin-6-yl)-1-(4-fluorobenzyl)-5-methyl-3-m-tolyl-1H-imidazol-2(3H)-one

The title compound was prepared in 60% yield, as per 4-methyl-3-(4-methylbenz-yl)-1-m-tolyl-5-[1,2,4]triazolo[1,5-a]pyridin-6-yl-1,3-dihydro-imidazol-2-one, substituting α-bromo-4-fluorotoluene (20 uL, 0.16 mmol, Aldrich) for 1-Bromomethyl-4-methylbenzene.

¹H NMR (500 MHz, DMSO-d6) δ ppm: 8.933 (s, 1H) 8.490 (s, 1H) 7.671 (d, J=9.3 Hz, 1H) 7.417 (m, 2H) 7.248-7.126 (m, 5H) 7.060 (d, J=7.7 Hz, 1H) 6.887 (d, J=8.1 Hz, 1H) 4.937 (s, 2H) 2.239 (s, 3H) 2.100 (s, 3H). (ESI (+) m/z): 414.34 (M+1).

### Example 39

### 4-((4-([1,2,4]triazolo[1,5-a]pyridin-6-yl)-5-methyl-2-oxo-3-m-tolyl-2,3-dihydro-1H-imidazol-1-yl)methyl)benzonitrile

The title compound was prepared in 53% yield, as per 4-methyl-3-(4-methyl-benz-yl)-1-m-tolyl-5-[1,2,4]triazolo[1,5-a]pyridin-6-yl-1,3-dihydro-imidazol-2-one, substituting p-cyanobenzyl bromide (0.036 g, 0.18 mmol, Aldrich) for 1-bromomethyl-4-methylbenzene as the electrophile.

¹H NMR (500 MHz, DMSO-d6) δ ppm: 8.954 (s, 1H) 8.500 (s, 1H) 7.877 (d, J=7.7 Hz, 2H) 7.683 (d, J=9.5 Hz, 1H) 7.538 (d, J=8.2 Hz, 2H) 7.226-7.133 (m, 3H) 7.067 (d, J=7.5 Hz, 1H) 6.901 (d, J=7.7 Hz, 1H) 5.056 (s, 2H) 2.239 (s, 3H) 2.076 (s, 3H). (ESI (+) m/z): 421.36 (M+1).

### Example 40

### 4-([1,2,4]triazolo[1,5-a]pyridin-6-yl)-1-(2-fluorobenzyl)-5-methyl-3-m-tolyl-1H-imidazol-2(3H)-one

The title compound was prepared as per 4-methyl-3-(4-methyl-benzyl)-1-m-tolyl-5-[1,2,4]triazolo[1,5-a]pyridin-6-yl-1,3-dihydro-imidazol-2-one, substituting 1-bromomethyl-2-fluoro-benzene (20 uL, 0.16 mmol; Aldrich) for 1-bromomethyl-4-methylbenzene.

¹H NMR (500 MHz, DMSO-d6) δ ppm: 8.956 (s, 1H) 8.500 (s, 1H) 7.685 (d, J=9.2 Hz, 1H) 7.380 (dd, J=7.5, 6.5 Hz, 1H) 7.320 (dd, J=7.5, 7.5 Hz, 1H) 7.292-7.205 (m, 2H) 7.201-7.152 (m, 2H) 7.141 (s, 1H) 7.061 (d, J=7.5 Hz, 1H) 6.893 (d, J=8.0 Hz, 1H) 4.996 (s, 2H) 2.237 (s, 3H) 2.103 (s, 3H). (ESI (+) m/z): 414.45 (M+1).

### Example 41

### 4-([1,2,4]triazolo[1,5-a]pyridin-6-yl)-1-(cyclopentylmethyl)-5-methyl-3-m-tolyl-1H-imidazol-2(3H)-one

4-Methyl-1-m-tolyl-5-[1,2,4]triazolo[1,5-a]pyridin-6-yl-1,3-dihydro-imidazol-2-one (53 mg, 0.17 mmol) was dissolved in N,N-dimethylformamide (3.0 mL) and cooled to 0° C. Potassium tert-butoxide (53 mg, 0.47 mmol, Aldrich) was added followed by iodomethyl-cyclopentane (0.059 g, 0.28 mmol, Acros). The reaction was heated to 80° C. and stirred at for 2 h. The reaction was cooled and purified by preparative HPLC to yield the product.

¹H NMR (500 MHz, DMSO-d6) δ ppm: 8.911 (s, 1H) 8.495 (s, 1H) 7.672 (d, J=9.0 Hz, 1H) 7.205-7.081 (m, 3H) 7.041 (d, J=7.6 Hz, 1H) 6.844 (d, J=8.2 Hz, 1H) 3.602 (d, J=7.6 Hz, 1H) 2.290 (m, 1H) 2.228 (s, 3H) 2.180 (s, 3H) 1.719-1.592 (m, 4H) 1.568-1.470 (m, 2H) 1.378-1.270 (m, 2H). (ESI (+) m/z): 388.42 (M+1).

### Example 42

### 4-([1,2,4]triazolo[1,5-a]pyridin-6-yl)-1-(cyclopropylmethyl)-5-methyl-3-m-tolyl-1H-imidazol-2(3H)-one

The title compound was prepared as per 1-cyclopentylmethyl-5-methyl-3-m-tolyl-4-[1,2,4]triazolo[1,5-a]pyridin-6-yl-1,3-dihydro-imidazol-2-one, substituting cyclopropylmethyl bromide (21 uL, 0.21 mmol, Aldrich) for iodomethylcyclopentane.

¹H NMR (500 MHz, DMSO-d6) δ ppm: 8.922 (s, 1H) 8.498 (s, 1H) 7.677 (d, J=9.3 Hz, 1H) 7.179-7.149 (m, 2H) 7.108 (s, 1H) 7.043 (d, J=7.5 Hz, 1H) 6.856 (d, J=8.0 Hz, 1H) 3.577 (d, J=6.7 Hz, 2H) 2.352 (m, 1H) 2.229 (s, 3H) 2.208 (s, 3H) 0.515 (d, J=7.7 Hz, 2H) 0.384 (d, J=4.3 Hz, 2H). (ESI (+) m/z): 360.21 (M+1).

### Example 43

### 4-([1,2,4]triazolo[1,5-a]pyridin-6-yl)-1-benzoyl-3-(3-chloro-4-fluorophenyl)-5-methyl-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (51 mg, 0.15 mmol) was dissolved in N,N-dimethylformamide (3.0 mL, Acros) and cooled to 0° C. Potassium tert-butoxide (46 mg, 0.41 mmol, Aldrich) was added and then benzoyl chloride (20 uL, 0.17 mmol, Aldrich). The reaction was allowed to warm to room temperature and stir for 3.5 hours. The material was purified by preparative HPLC to yield the product.

¹H NMR (500 MHz, DMSO-d6) δ ppm: 9.119 (s, 1H) 8.571 (s, 1H) 7.951 (d, J=7.8 Hz, 2H) 7.798 (d, J=9.9 Hz, 1H) 7.731-7.664 (m, 2H) 7.562 (dd, J=7.5, 7.5 Hz, 2H) 7.366 (dd, J=9.0, 9.0 Hz, 1H) 7.296 (d, J=9.0 Hz, 1H) 7.196 (m, 1H) 2.244 (s, 3H). (ESI (+) m/z): 448.21 (M+1).

### Example 44

### 4-([1,2,4]triazolo[1,5-a]pyridin-6-yl)-3-(3-chloro-4-fluorophenyl)-5-methyl-1-(methylsulfonyl)-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50 mg, 0.0001 mol.) was dissolved in N,N-dimethylformamide (3 mL, 0.04 mol.). Potassium tert-butoxide (0.023 g, 0.00020 mol.) was added then methanesulfonyl chloride (12 uL, 0.00016 mol.). The reaction was allowed to stir at room temperature for 1-2 hours. LC-MS showed formation of product at 1.35/422.13. The product was isolated by Gilson preparative HPLC.

¹H NMR (300 MHz, DMSO-d6) δ ppm: 9.06, m, 1H, 8.69, m, 1H, 8.56, s, 1H, 7.79, d, J=9 Hz, 1H, 7.69, m, 1H, 7.39, t, J=9 Hz, 1H, 7.28, m, 1H, 7.21, m, 1H, 2.64, s, 3H, 2.16, s, 3H. (ESI (+) m/z): 422.13 (M+H+)

### Example 45

### 4-([1,2,4]triazolo[1,5-a]pyridin-6-yl)-3-(3-chloro-4-fluorophenyl)-5-methyl-1-(phenylsulfonyl)-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50 mg, 0.0001 mol.) was dissolved in N,N-dimethylformamide (3 mL, 0.04 mol.). Potassium tert-butoxide (0.023 g, 0.00020 mol.) was added then benzenesulfonyl chloride (20.4 uL, 0.000160 mol.). The reaction was stirred at room temperature for 1-2 hours. LC-MS showed formation of product at 1.64/484.16. The product was isolated by Gilson preparative HPLC.

¹H NMR (300 MHz, DMSO-d6) δ ppm: 9.09, m, 1H, 8.55, s, 1H, 8.15, m, 2H, 7.85, m, 1H, 7.79-7.70, m, 3H, 7.60, m, 1H, 7.35-7.29, m, 2H, 7.13, m, 1H, 2.36, s, 3H. (ESI (+) m/z): 484.16 (M+H+)

### Example 46

### 4-([1,2,4]triazolo[1,5-a]pyridin-6-yl)-1-acetyl-3-(3-chloro-4-fluorophenyl)-5-methyl-1H-imidazol-2(3H)-one

1-(3-Chloro-4-fluoro-phenyl)-4-methyl-5-[1,2,4]triazolo[1,5-a]pyridin-6-yl-1,3-dihydro-imidazol-2-one (50 mg, 0.0001 mol.) was dissolved in N,N-dimethylformamide (3 mL, 0.04 mol.). Potassium tert-butoxide (0.023 g, 0.00020 mol.) was added then acetic anhydride (15 uL, 0.00016 mol.). The reaction was allowed to stir at room temperature for 1-2 hours. LC-MS showed formation of product at 1.42/386.17. The product was isolated by Gilson preparative HPLC.

¹H NMR (300 MHz, DMSO-d6) δ ppm: 9.07, s, 1H, 8.56, s, 1H, 7.79, d, J=10 Hz, 1H, 7.68, m, 1H, 7.39, t, J=10 Hz, 1H, 7.29, m, 1H, 7.21, m, 1H, 2.65, s, 3H, 2.30, s, 3H. (ESI (+) m/z): 386.17 (M+H+)

### Example 47

### Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβ. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μL of 1.25 μCi ³³P-ATP/25 μM ATP in assay buffer (50 mM Hepes, 60 mM NaCl, 1 mM MgCl₂, 2 mM DTT, 5 mM MnCl₂, 2% glycerol, and 0.015% Brij® 35). 10 μL of each test compound of Formula (I) prepared in 5% DMSO solution were added to the FlashPlate. The assay was then initiated with the addition of 20 μL of assay buffer containing 12.5 μmol of His-TGFβRI to each well. Plates were incubated for 30 minutes at room temperature and the reactions were then terminated by a single rinse with TBS. Radiation from each well of the plates was read on a TopCount (Packard). Total binding (no inhibition) was defined as counts measured in the presence of DMSO solution containing no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of Formula (I) typically exhibited IC₅₀ values of less than 10 μM; some exhibited IC₅₀ values of less than 1 μM; and some even exhibited IC₅₀ values of less than 50 nM.

### Example 48

### Cell-Free Assay for Evaluating Inhibition of Activin Type I Receptor Kinase Activity

Inhibition of the Activin type I receptor (Alk 4) kinase autophosphorylation activity by test compounds of Formula (I) can be determined in a similar manner to that described above in Example 34 except that a similarly His-tagged form of Alk4 (His-Alk4) is used in place of the His-TGFβRI.

### Example 49

### TGFβ Type I Receptor Ligand Displacement FlashPlate Assay

50 nM of tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Bridj® 35; pH 7.5) was premixed with a test compound of Formula (I) in 1% DMSO solution in a v-bottom plate. Control wells containing either DMSO without any test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to a nickel coated FlashPlate (PE, NEN catalog number: SMP107), while the control wells contained only buffer (i.e., no His-TGFβ Type I receptor). The premixed solution of tritiated 4-(3-pyridin-2-yl-1H-pyrazol-4-yl)-quinoline and test compound of Formula (I) was then added to the wells. The wells were aspirated after an hour at room temperature and radioactivity in wells (emitted from the tritiated compound) was measured using TopCount (PerkinElmer LifeSciences, Inc., Boston Mass.).

Compounds of Formula (I) typically exhibited Kᵢ values of less than 10 μM; some exhibited Kᵢ values of less than 1 μM; and some even exhibited Kᵢ values of less than 50 nM.

### Example 50

### Assay for Evaluating Cellular Inhibition of TGFβ Signaling and Cytotoxicity

Biological activity of the compounds of Formula (I) was determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/mL), streptomycin (100 μg/mL), L-glutamine (2 mM), sodium pyruvate (1 mM), and non-essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with 2.5 ng/mL TGFβ ligand in the starvation media containing 1% DMSO either in the presence or absence of a test compound of Formula (I) and incubated as described above for 24 hours. The media was washed ou t the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of Formula (I) for inhibiting TGFβ-induced PAI-Luciferase reporter activity in HepG2 cells. Compounds of Formula (I) typically exhibited IC₅₀ values of less 10 μM. Cytotoxicity was determined using the same cell culture conditions as described above. Specifically, cell viability was determined after overnight incubation with the CytoLite cell viability kit (Packard, Cat. No. 6016901). Compounds of Formula (I) typically exhibited LD₂₅ values greater than 10 μM.

### Example 51

### Assay for Evaluating Inhibition of TGFβ Type I Receptor Kinase Activity in Cells

The cellular inhibition of activin signaling activity by the test compounds of Formula (I) is determined in a similar manner as described above in Example 37 except that 100 ng/mL of activin is added to serum starved cells in place of the 2.5 ng/mL TGFβ.

### Example 52

### Assay for TGFβ-Induced Collagen Expression

### Preparation of Immortalized Collagen Promotor-Green Fluorescent Protein Cells

Fibroblasts are derived from the skin of adult transgenic mice expressing Green Fluorescent Protein (GFP) under the control of the collagen 1A1 promoter (see Krempen, K. et al., Gene Exp. 8: 151-163 (1999)). Cells are immortalized with a temperature sensitive large T antigen that is in an active stage at 33° C. Cells are expanded at 33° C. and then transferred to 37° C. at which temperature the large T antigen becomes inactive (see Xu, S. et al., Exp. Cell Res. 220: 407-414 (1995)). Over the course of about 4 days and one split, the cells cease proliferating. Cells are then frozen in aliquots sufficient for a single 96 well plate.

### Assay of TGFβ-Induced Collagen-GFP Expression

Cells are thawed, plated in complete DMEM (contains non-essential amino acids, 1 mM sodium pyruvate and 2 mM L-glutamine) with 10% fetal calf serum, and then incubated for overnight at 37° C., 5% CO₂. The cells are trypsinized in the following day and transferred into 96 well format with 30,000 cells per well in 50 μL complete DMEM containing 2% fetal calf serum, but without phenol red. The cells are incubated at 37° C. for 3 to 4 hours to allow them to adhere to the plate. Solutions containing a test compound of Formula (I) are then added to wells with no TGFβ (in triplicates), as well as wells with 1 ng/mL TGFβ (in triplicates). DMSO is also added to all of the wells at a final concentration of 0.1%. GFP fluorescence emission at 530 nm following excitation at 485 nm is measured at 48 hours after the addition of solutions containing a test compound on a CytoFluor microplate reader (PerSeptive Biosystems). The data are then expressed as the ratio of TGFβ-induced to non-induced for each test sample.

### Example 53

### Fluorescence Polarization Assay for Evaluating Inhibition of TGFβ Receptor

Competitive displacement using a fluorescence polarization assay utilized an Oregon green-labeled ALK4/5 inhibitor, which was shown to bind with high affinity to ALK5 (Kd, 0.34+0.01 nmol/L) and ALK4 (Kd, 0.53+0.03 nmol/L), using fluorescence polarization saturation curve analysis. Varying concentrations of compounds of Formula (I) and 25 nmol/L of the Oregon Green-labeled ALK4/5 inhibitor were incubated (1 hour, room temperature, in the dark) with 4.5 nmol/L of hALK4-K or hALK5-K, 30 mmol/L Hepes pH 7.5, 20 mmol/L NaCl, 1 mmol/L MgCl₂, 100 mmol/L KCl, 0.01% BSA, 0.01% Tween-20 at a final concentration of 1% DMSO in black 96-well Microfluor 2 plates (Cat. No. 7205, ThermoLab Systems).

The signal was detected at excitation/emission settings of 490/530 nanometers using an Analyst HT (LJL BioSystems, Sunnyvale, Calif.). The IC₅₀ values for the tested compounds of Formula (I) were determined by nonlinear regression and their Ki values were calculated from the Cheng-Prusoff equation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-52. (canceled)

53. A compound of Formula (I), 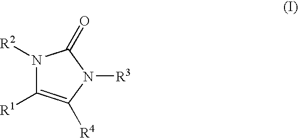
an N-oxide derivative thereof, or a pharmaceutically acceptable salt thereof, wherein:
R¹ is an optionally substituted monocyclic heteroaryl containing at least one hetero ring atom selected from the group consisting of O and S, and optionally further containing 1 or 2 N atoms as hetero ring atoms; or R¹ is an optionally substituted monocyclic heteroaryl containing at least 3 N atoms as hetero ring atoms; or R¹ is an optionally substituted 9- to 12-membered bicyclic heteroaryl containing at least 1 ring atom selected from the group consisting of O and S, and optionally also containing 1 to 3 N atoms as hetero ring atoms; or R¹ is an optionally substituted 9- to 12-membered bicyclic heteroaryl containing at least 2 ring atoms each independently selected from the group consisting of O, S, and N; or R¹ is an optionally substituted 10- to 12-membered bicyclic heteroaryl containing at least 1 ring atom each independently selected from the group consisting of O, S, and N;
R² is an optionally substituted aryl or an optionally substituted heteroaryl;
R³ is selected from the group consisting of H, optionally substituted aliphatic, optionally substituted cycloaliphatic, optionally substituted heterocycloaliphatic, optionally substituted araliphatic, optionally substituted heteroaraliphatic, optionally substituted aryl, and optionally substituted heteroaryl; and
R⁴ is selected from the group consisting of H, optionally substituted aliphatic, optionally substituted cycloaliphatic, optionally substituted heterocycloaliphatic, optionally substituted araliphatic, optionally substituted heteroaraliphatic, optionally substituted aryl, and optionally substituted heteroaryl.

54. The compound of claim 53, wherein R¹ is benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, or cinnolinyl; and R¹ is optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

55. The compound of claim 54, wherein R¹ is optionally substituted [1,2,4]triazolo[1,5-a]pyridin-6-yl.

56. The compound of claim 55, wherein R² is aryl optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

57. The compound of claim 56, wherein R² is phenyl, methylphenyl or chlorophenyl, fluorophenyl or chlorofluoropheny

58. The compound of claim 56 wherein R² is selected from the group consisting of benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, and cinnolinyl; and R² is optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

59. The compound of claim 53, wherein R³ is selected from the group consisting of H, optionally substituted C₁₋₆ aliphatic, optionally substituted C₃₋₁₀ cycloaliphatic, optionally substituted C₃₋₁₀ heterocycloaliphatic, optionally substituted C₄₋₁₀ araliphatic, optionally substituted C₃₋₁₀ heteroaraliphatic, optionally substituted C₄₋₁₀ aryl, and optionally substituted C₃₋₁₀ heteroaryl.

60. The compound of claim 59, wherein R³ is methyl substituted with an optionally substituted aryl or an optionally substituted heteroaryl.

61. The compound of claim 60, wherein R³ is benzyl and the phenyl moiety in the benzyl group is optionally substituted with 1 to 3 substituents each independently selected from the group consisting of alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkllsulfanyl, heterocycloalkylcarbonyl, aryl, aryloxy, arylsulfanyl, aroyl, heteroaryl, heteroaryloxy, heteroarylsulfanyl, and heteroaroyl.

62. The compound of claim 61, wherein R³ is methyl substituted with a heteroaryl selected from the group consisting of benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5-a]pyridinyl, [1,2,4]triazolo[4,3-a]pyridinyl, quinolinyl, quinoxalinyl, quinazolinyl, isoquinolinyl, and cinnolinyl; wherein the heteroaryl is optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

63. The compound of claim 53, wherein R³ is an optionally substituted cycloaliphatic of Formula (I)a, 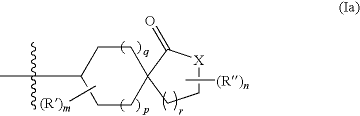
wherein
X is O or NR^{Q};
R^{Q} is H, C₁₋₄ aliphatic, C₃₋₇ cycloalkyl, C₆₋₁₂ aryl, or C₅₋₁₂ heteroaryl;
each R′ is independently C₁₋₄ aliphatic, halo, cyano, hydroxy, carboxy, amido, amino, or alkoxy;
each R″ is independently C₁₋₄ aliphatic, halo, cyano, hydroxy, carboxy, amido, amino, or alkoxy;
each of p and q is independently 0, 1, or 2, provided that the sum of p and q is 2, 3, or 4;
r is 1, 2 or 3; and
each of m and n is independently 0, 1, or 2.

64. A compound of claim 63 wherein R₄ is H or C₁₋₆ aliphatic.

65. A compound of Formula (I), 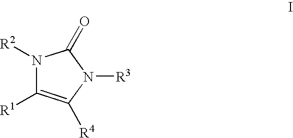
an N-oxide derivative thereof, or a pharmaceutically acceptable salt thereof, wherein:
R¹ is an optionally substituted [1,2,4]triazolo[1,5-a]pyridinyl;
R² is an optionally substituted phenyl;
R³ is selected from the group consisting of H, optionally substituted aliphatic, optionally substituted cycloaliphatic, optionally substituted heterocycloaliphatic, optionally substituted araliphatic and optionally substituted heteroaraliphatic; and
R⁴ is H or C₁₋₆ alkyl.

66. The compound of claim 65, wherein phenyl is substituted by one or two substitutents selected from C₁₋₆ alkyl, chloro or fluoro.

67. The compound of claim 65, wherein R³ is C₁₋₆ alkyl, 1-oxo-2-oxaspiro[4.5]decan-8-yl, methylsulfonyl, or phenylsulfonyl; wherein C₁₋₆ alkyl, is optionally substituted with C₃₋₁₀ cycloaliphatic, oxo, alkoxy, carboxalkyl, alkoxycarbonylalkyl or phenyl optionally with 1 to 3 substituents each independently selected from the group consisting of C₁₋₆ alkyl, chloro or fluoro, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

68. The compound is selected from:
3-((4-([1,2,4]triazolo[1,5-a]pyridin-6-yl)-5-methyl-2-oxo-3-m-tolyl-2,3-dihydro-1H-imidazol-1-yl)methyl)benzoic acid;
4-((4-([1,2,4]triazolo[1,5-a]pyridin-6-yl)-5-methyl-2-oxo-3-m-tolyl-2,3-dihydro-1H-imidazol-1-yl)methyl)benzoic acid;
5-([1,2,4]triazolo[1,5-a]pyridin-6-yl)-1-(3-chloro-4-fluorophenyl)-4-methyl-1H-imidazol-2(3H)-one;
4-([1,2,4]triazolo[1,5-a]pyridin-6-yl)-5-methyl-1-(1-oxo-2-oxaspiro[4.5]decan-8-yl)-3-m-tolyl-1H-imidazol-2(3H)-one;
4-([1,2,4]triazolo[1,5-a]pyridin-6-yl)-1-(cyclobutylmethyl)-5-methyl-3-phenyl-1H-imidazol-2(3H)-one;
4-([1,2,4]triazolo[1,5-a]pyridin-6-yl)-1-(2-aminobenzyl)-5-methyl-3-m-tolyl-1H-imidazol-2(3H)-one;
4-([1,2,4]triazolo[1,5-a]pyridin-6-yl)-1-(benzo[c][1,2,5]thiadiazol-5-ylmethyl)-5-methyl-3-m-tolyl-1H-imidazol-2(3H)-one;
4-([1,2,4]triazolo[1,5-a]pyridin-6-yl)-5-methyl-1-(2-methylbenzyl)-3-m-tolyl-1H-imidazol-2(3H)-one;
4-([1,2,4]triazolo[1,5-a]pyridin-6-yl)-5-methyl-1-(2-nitrobenzyl)-3-m-tolyl-1H-imidazol-2(3H)-one;
2-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]triazolo[1,5-a]pyridin-6-yl)-1-(3-methoxybenzyl)-5-methyl-3-m-tolyl-1H-imidazol-2(3H)-one;
4-([1,2,4]triazolo[1,5-a]pyridin-6-yl)-5-methyl-1-(3-methylbenzyl)-3-m-tolyl-1H-imidazol-2(3H)-one;
4-([1,2,4]triazolo[1,5-a]pyridin-6-yl)-1-(3-fluorobenzyl)-5-methyl-3-m-tolyl-1H-imidazol-2(3H)-one;
3-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]thiazolo[1,5-a]pyridin-6-yl)-5-methyl-1-((tetrahydro-2H-pyran-4-yl)methyl)-3-m-tolyl-1H-imidazol-2(3H)-one;
4-([1,2,4]triazolo[1,5-a]pyridin-6-yl)-5-methyl-1-((5-methylisoxazol-3-yl)methyl)-3-m-tolyl-1H-imidazol-2(3H)-one;
4-([1,2,4]triazolo[1,5-a]pyridin-6-yl)-1-(benzo[c][1,2,5]oxadiazol-5-ylmethyl)-5-methyl-3-m-tolyl-1H-imidazol-2(3H)-one;
ethyl 2-(4-([1,2,4]triazolo[1,5-a]pyridin-6-yl)-5-methyl-2-oxo-3-m-tolyl-2,3-dihydro-1H-imidazol-1-yl)acetate;
4-([1,2,4]triazolo[1,5-a]pyridin-6-yl)-1-(2-methoxyethyl)-5-methyl-3-m-tolyl-1H-imidazol-2(3H)-one;
4-([1,2,4]triazolo[1,5-a]pyridin-6-yl)-5-methyl-1-(pyridin-3-ylmethyl)-3-m-tolyl-1H-imidazol-2(3H)-one;
4-([1,2,4]triazolo[1,5-a]pyridin-6-yl)-5-methyl-1-(pyridin-2-ylmethyl)-3-m-tolyl-1H-imidazol-2(3H)-one;
4-([1,2,4]triazolo[1,5-a]pyridin-6-yl)-1-(3-aminobenzyl)-5-methyl-3-m-tolyl-1H-imidazol-2(3H)-one;
4-([1,2,4]triazolo[1,5-a]pyridin-6-yl)-1-(cyclohexylmethyl)-5-methyl-3-m-tolyl-1H-imidazol-2(3H)-one;
4-([1,2,4]triazolo[1,5-a]pyridin-6-yl)-1,5-dimethyl-3-m-tolyl-1H-imidazol-2(3H)-one;
3-((4-([1,2,4]triazolo[1,5-a]pyridin-6-yl)-5-methyl-2-oxo-3-m-tolyl-2,3-dihydro-1H-imidazol-1-yl)methyl)benzamide;
methyl 3-((4-([1,2,4]triazolo[1,5-a]pyridin-6-yl)-5-methyl-2-oxo-3-m-tolyl-2,3-dihydro-1H-imidazol-1-yl)methyl)benzoate;
4-([1,2,4]triazolo[1,5-a]pyridin-6-yl)-5-methyl-1-(3-nitrobenzyl)-3-m-tolyl-1H-imidazol-2(3H)-one;
methyl 4-((4-([1,2,4]triazolo[1,5-a]pyridin-6-yl)-5-methyl-2-oxo-3-m-tolyl-2,3-dihydro-1H-imidazol-1-yl)methyl)benzoate;
4-([1,2,4]triazolo[1,5-a]pyridin-6-yl)-1-benzyl-5-methyl-3-m-tolyl-1H-imidazol-2(3H)-one;
4-([1,2,4]triazolo[1,5-a]pyridin-6-yl)-1-(4-methoxybenzyl)-5-methyl-3-m-tolyl-1H-imidazol-2(3H)-one;
5-([1,2,4]triazolo[1,5-a]pyridin-6-yl)-4-methyl-1-m-tolyl-1H-imidazol-2(3H)-one;
5-([1,2,4]triazolo[1,5-a]pyridin-6-yl)-1-m-tolyl-1H-imidazol-2(3H)-one;
4-((4-([1,2,4]triazolo[1,5-a]pyridin-6-yl)-5-methyl-2-oxo-3-m-tolyl-2,3-dihydro-1H-imidazol-1-yl)methyl)benzoic acid;
4-([1,2,4]triazolo[1,5-a]pyridin-6-yl)-3-(3-chloro-4-fluorophenyl)-5-methyl-1-(3-methylbenzyl)-1H-imidazol-2(3H)-one;
4-([1,2,4]triazolo[1,5-a]pyridin-6-yl)-1-(benzo[c][1,2,5]oxadiazol-5-ylmethyl)-3-(3-chloro-4-fluorophenyl)-5-methyl-1H-imidazol-2(3H)-one;
4-([1,2,4]triazolo[1,5-a]pyridin-6-yl)-1-(benzo[c][1,2,5]thiadiazol-5-ylmethyl)-3-(3-chloro-4-fluorophenyl)-5-methyl-1H-imidazol-2(3H)-one;
4-([1,2,4]triazolo[1,5-a]pyridin-6-yl)-3-(3-chloro-4-fluorophenyl)-1-(3-fluorobenzyl)-5-methyl-1H-imidazol-2(3H)-one;
4-([1,2,4]triazolo[1,5-a]pyridin-6-yl)-5-methyl-1-(4-methylbenzyl)-3-m-tolyl-1H-imidazol-2(3H)-one;
4-([1,2,4]triazolo[1,5-a]pyridin-6-yl)-1-(4-fluorobenzyl)-5-methyl-3-m-tolyl-1H-imidazol-2(3H)-one;
4-((4-([1,2,4]triazolo[1,5-a]pyridin-6-yl)-5-methyl-2-oxo-3-m-tolyl-2,3-dihydro-1H-imidazol-1-yl)methyl)benzonitrile;
4-([1,2,4]triazolo[1,5-a]pyridin-6-yl)-1-(2-fluorobenzyl)-5-methyl-3-m-tolyl-1H-imidazol-2(3H)-one;
4-([1,2,4]triazolo[1,5-a]pyridin-6-yl)-1-(cyclopentylmethyl)-5-methyl-3-m-tolyl-1H-imidazol-2(3H)-one;
4-([1,2,4]triazolo[1,5-a]pyridin-6-yl)-1-(cyclopropylmethyl)-5-methyl-3-m-tolyl-1H-imidazol-2(3H)-one;
4-([1,2,4]triazolo[1,5-a]pyridin-6-yl)-1-benzoyl-3-(3-chloro-4-fluorophenyl)-5-methyl-1H-imidazol-2(3H)-one;
4-([1,2,4]triazolo[1,5-a]pyridin-6-yl)-3-(3-chloro-4-fluorophenyl)-5-methyl-1-(methylsulfonyl)-1H-imidazol-2(3H)-one;
4-([1,2,4]triazolo[1,5-a]pyridin-6-yl)-3-(3-chloro-4-fluorophenyl)-5-methyl-1-(phenylsulfonyl)-1H-imidazol-2(3H)-one; or
4-([1,2,4]triazolo[1,5-a]pyridin-6-yl)-1-acetyl-3-(3-chloro-4-fluorophenyl)-5-methyl-1H-imidazol-2(3H)-one.

69. A pharmaceutical composition comprising a compound of claim 53 and a pharmaceutically acceptable carrier.

70. A method selected from any of the following methods, inhibiting the TGFβ signaling pathway in a subject, inhibiting the TGFβ type I receptor in a cell, reducing the accumulation of excess extracellular matrix induced by TGFβ in a subject, treating or preventing a fibrotic condition; inhibiting growth or metastasis of tumor cells or cancer in a subject, treating carcinomas or any diseases mediated by an overexpression of TGFβ, treating or preventing restinosis, vascular disease, or hypertension, comprising administering to the subject in need thereof an effective amount of a compound of claim 53.

71. The method of claim 70, wherein the fibrotic condition is selected from the group consisting of scleroderma, lupus nephritis, connective tissue disease, wound healing, surgical scarring, spinal cord injury, CNS scarring, acute lung injury, idiopathic pulmonary fibrosis, radiation-induced pulmonary fibrosis, chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, fatty liver disease, primary sclerosing cholangitis, restenosis, radiation-induced fibrosis, chemotherapy-induced fibrosis, cardiac fibrosis, opthalmic scarring, fibrosclerosis, a fibrotic cancer, a fibroid, fibroma, a fibroadenoma, a fibrosarcoma, transplant arteriopathy, mesothelioma, and keloid.

72. The method of claim 70, wherein said carcinomas are selected from the group consisting of carcinomas of the lung, breast, liver, biliary tract, gastrointestinal tract, head and neck, pancreas, prostate, cervix, multiple myeloma, melanoma, glioma, and glioblastomas; or
wherein the disease or disorder is selected from the group consisting of demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, head, and neck; or
wherein said restinosis coronary restenosis, peripheral restenosis, or carotid restenosis; or
wherein said vascular disease is intimal thickening, vascular remodeling, or organ transplant-related vascular disease; or
wherein said hypertension is primary or secondary hypertension, systolic hypertension, pulmonary hypertension, or hypertension-induced vascular remodeling.
